(19) United States
(12) Patent Application Publication
(10) Pub. No.: US 2011/0159527 A1
(43) Pub. Date: Jun. 30, 2011

(54) METHODS AND KITS FOR DIAGNOSING NEURODEGENERATIVE DISEASE

(76) Inventors: Michael Gebhard Schlossmacher, Ottawa (CA); Brit Mollenhauer, Kassel (DE); Omar M.A. El Agnaf, Al-Ain (AE)

(21) Appl. No.: 12/996,604

(22) PCT Filed: Jun. 15, 2009
(86) PCT No.: PCT/CA2009/000822

§ 371 Date: Mar. 11, 2011

Related U.S. Application Data
(60) Provisional application No. 61/061,914, filed on Jun. 16, 2008.

(51) Int. Cl. G01N 33/53; G01N 33/68; C12Q 1/02

(52) U.S. Cl. 435/7.92; 436/86; 435/29; 436/501

(57) ABSTRACT

Methods and diagnostic kits for determining whether a subject may develop a or for diagnosing a neurodegenerative disease. The method includes quantitating the amount of alpha-synuclein and total protein in a cerebrospinal fluid (CSF) sample obtained from the subject and calculating a ratio of alpha-synuclein to total protein content; comparing the ratio of alpha-synuclein to total protein content in the CSF sample with the alpha-synuclein to total protein content ratio in CSF samples obtained from healthy neurodegenerative disease-free subjects; and (c) determining from the comparison whether the subject has a likelihood to develop neurodegenerative disease or making a diagnosis of neurodegenerative disease in a subject. A difference in the ratio of alpha-synuclein to total protein content indicates that the subject has a likelihood to develop a neurodegenerative disease or has developed a neurodegenerative disease.

### FIELD OF THE INVENTION

The invention relates to assays for the diagnosis of neurodegenerative diseases. In particular, the invention relates to a biochemical method of correlating changes in the biological abundance of alpha-synuclein in CSF and other biological samples to determine the likelihood of a subject developing a neurodegenerative disease, or for making or aiding in the diagnosis of neurodegenerative diseases.

### BACKGROUND OF THE INVENTION

Several diseases of the human brain result in progressive parkinsonism or dementia, or both. These include Parkinson disease (PD), dementia with Lewy bodies (DLB), multiple system atrophy (MSA), Alzheimer disease (AD) and Creutzfeldt-Jakob disease (CJD). Pathologically, these neurodegenerative disorders often feature characteristic protein aggregates. Synucleinopathies share the deposition of insoluble alpha-synuclein (α-synuclein; αS) within neurons (as in PD, DLB) or oligodendrocytes (as in MSA) (Gai et al., 1998, Halliday and McCann, 2008, Jellinger, 2003 and Spillantini et al., 1997). In AD and CJD, distinct cerebrospinal fluid (CSF) markers have been developed and validated to complement the clinical diagnosis (Dubois et al., 2007 and Fagan et al., 2007). For synucleinopathies, no protein-based laboratory markers have been established to date that could assist in their differentiation from other conditions (Blennow and Hampel, 2003, Dorsey et al., 2006, Klein and Schlossmacher, 2006, Michell et al., 2004 and Scherzer et al., 2007). Encoded by the SNCA gene, alpha-synuclein is an abundant intracellular protein. Missense mutations and SNCA gene multiplication events have been linked to heritable forms of parkinsonism, dementia and autonomic dysfunction (Polymeropoulos et al., 1997 and Singleton et al., 2003) (reviewed in Farrer, 2006 and Schlossmacher, 2007). It is believed that the dynamics of alpha-synuclein homeostasis can be altered by different events, thereby promoting disease (Cookson, 2005 and Cookson and van der Brug, 2008); these include an enhanced synthesis rate (Miller D W et al., Neurology 2004) Outeiro and Lindquist, 2003 and Singleton et al., 2003), increased propensity for mutants to generate protofibrils (Conway et al., 2000 and El-Agnaf et al., 1998), sustained phosphorylation at Ser129 and C-terminal truncation (Anderson et al., 2006), as well as reduced degradation rates (Cuervo et al., 2004; Cullen V et al., Mol Brain 2009). Recent work has focused on proteasomal and lysosomal pathways for alpha-synuclein turnover (Cuervo et al., 2004, Lee et al., 2005, Shin et al., 2005 and Stefanis et al., 2001), but the third method of lowering intracellular alpha-synuclein may be through its release into the extracellular space (El-Agnaf et al., 2003). Full-length alpha-synuclein has been previously detected in biological fluids, including plasma and conditioned cell media, but evidence for its release as a physiological event has not yet been provided (El-Agnaf et al., 2003, Lee et al., 2005 and Li et al., 2007).

Several investigators have reasoned that the quantification of extracellular alpha-synuclein could provide a platform for marker development for human synucleinopathies (El-Agnaf et al., 2006, Fjorback et al., 2007, Tokuda et al., 2006 and van Geel et al., 2008).

Recently, the quantification of CSF alpha-synuclein from living donors by a first-generation, enzyme-linked immunoadsorbent assay (ELISA) (Tokuda et al., 2006) was explored, which revealed lower concentrations in 33 PD patients in a cross-sectional study of 71 subjects. However, five issues were identified that could have affected the study's conclusions and wider applicability of the method: one, alpha-synuclein protein(s) in human CSF have never been characterized by sequencing methods; two, CSF alpha-synuclein reactivity could also result from low level contamination by blood products (El-Agnaf et al., 2003, Li et al., 2007 and Miller et al., 2004); three, the concentration of αS in relation to the total CSF protein concentration and the plasma αS concentration had not been determined; four, CSF had to be concentrated five-fold prior to loading (Tokuda et al., 2006), thereby potentially leading to non-uniform protein concentration variability; and five, donors with neurodegenerative syndromes other than PD had not been analyzed in earlier studies using an ELISA that is directed to αS.

While other imaging and laboratory-based markers have been employed to support the clinical diagnosis of neurodegenerative diseases (or to assess its rate of progression), their usefulness has often been limited by a variety of factors including—but not limited to—availability, cost, turn-around-time (e.g., fluorodopa PET studies), and lack of sensitivity and specificity. For example, CSF based biomarkers such as 14-3-3 protein, total tau (and phospho-specific tau), and amyloid β protein₁₋₄₂, lack specificity and sensitivity when it comes to identifying a specific neurodegenerative disease based on a single marker value, however, they can reach higher specificity and sensitivity when combined. The currently most extensively studied value set is the ratio of total tau to amyloid β protein₁₋₄₂ in the early detection of AD (Fagan A M et al., Ann Neurol 2009; Fagan A M et al., Arch Neurol 2007; Fagan A M et al., Ann Neurol 2006).

Accordingly, there remains no convenient, discriminatory and sensitive ex vivo laboratory method for the diagnosis of neurodegenerative diseases, such as PD, DLB, MSA and CJD as well as other alpha-synuclein and/or neuronal cell loss-related disorders.

### SUMMARY OF THE INVENTION

The present inventors have sought to address the above-described need through the development of a biochemical assay and algorithm for diagnosing several neurodegenerative diseases.

Accordingly, the invention relates to a method to determine whether a subject has a likelihood to develop a neurodegenerative disease (disease risk), or for diagnosing a neurodegenerative disease in said subject (disease state), or for diagnosing the rate of advance for a neurodegenerative disease in said subject (disease progression), characterized by the following steps:
(a) quantifying the amount of total alpha-synuclein (i.e., encompassing full-length alpha-synuclein, C- or N-terminally truncated alpha-synuclein species, monomeric and oligomeric forms, and post-translationally modified variants (such as lipid-associated, sugar-associated, protein-associated, nitrated, oxidized, acetylated or phosphorylated at any one of its residues) of CSF alpha-synuclein) (e.g. by sandwich ELISA) and of total protein (e.g. using the Bradford method or other established method) in a cerebrospinal fluid (CSF) sample obtained from said subject and calculating a ratio of total alpha-synuclein to total protein content (preferably the spinal fluid sample for the analyses will be clear of cellular contaminants, and will not represent an aliquot from within the first 15 cc of fluid collected by lumbar puncture);
(b) determining the age of the donor;
(c) comparing the ratio of alpha-synuclein to total protein content in said CSF sample obtained from said subject with the alpha-synuclein to total protein content ratio in CSF reference samples obtained from neurologically healthy subjects (and preferably medically healthy subjects) from the same age range that at the time of sampling did not show clinical signs of neurodegenerative disease; and
(d) determining, from the comparison in step (d), whether the subject has a likelihood to develop neurodegenerative disease or making a diagnosis of neurodegenerative disease in said subject, whereby a difference in the ratio of total alpha-synuclein to total protein content in the CSF sample of said subject when compared with the ratio of alpha-synuclein to total protein content in the CSF reference samples obtained from the neurologically healthy subjects from the same age range indicates that said subject has a likelihood to develop a neurodegenerative disease or has developed a neurodegenerative disease.

The levels of alpha-synuclein and total protein in CSF are subject to change in humans dependent on the age of the donor and independent upon any concomitant illness. Accordingly, age of the subject is an important factor to take into account. For example, yet without wishing to be limiting in any manner, in neurologically healthy human subjects the applicants have observed a steady increase throughout adulthood in the mean CSF protein concentration, i.e., from ≦0.59 mg/ml for a group of subjects aged 30-39 years to ≧0.69 mg/ml in a cohort of 70-79 year-old donors. Likewise, we have recorded an approximately 10 percent decrease per decade of ageing in the total concentration of CSF alpha-synuclein in healthy individuals older than 60 years.

In preferred embodiments of the above method, the amount of total tau protein in a cerebrospinal fluid CSF sample from said subject will be quantified. Tests for the tau protein are known and commercially available, such as the ELISA for tau available from Innogenetics. Quantifying tau in this manner is a helpful method of discriminating healthy aged controls from subjects with Alzheimer's and Alzheimer's-like diseases (referred to as tauopathies).

Either a reduction or an increase in the ratio of alpha-synuclein to total protein content in the CSF sample of the subject can be indicative of a neurological disorder. An approximately >1.5-fold reduction in the ratio of alpha-synuclein to total protein content in the CSF sample of the subject raises the possibility of a neurological disorder that is associated with intracellular synucleinopathy such as Parkinson disease, dementia with Lewy bodies or multiple system atrophy; an increase in the ratio of alpha-synuclein to total protein content by approximately >2.5 fold in the CSF sample of the subject raises the possibility of a neurological disorder that is associated with a relatively slow and more extensive neurodegenerative disease process than is seen in Parkinson's, such as in Alzheimer disease; an increase in the ratio of alpha-synuclein to total protein content by approximately >100-fold in the CSF sample of the subject raises the possibility of a neurological disorder that is associated with a nervous system-wide and more rapid neurodegenerative disease process such as in stroke and prion disease.

Accordingly, the above method may further include a step of:
(e) determining whether the subject has a likelihood to develop neurodegenerative disease or making a diagnosis of neurodegenerative disease within the group of Alzheimer disease and Alzheimer-like illnesses in a subject, whereby a rise in the content of total tau protein in the CSF sample of said subject when compared with the total tau level in the CSF samples obtained from neurologically healthy subjects from the same age range indicates that said subject has a likelihood to develop a neurodegenerative disease or has developed a neurodegenerative disease with Alzheimer disease-type pathology;

Analyses in applicants' recent biomarker study efforts (analyzing a cohort of n=319 donors) have shown that the mean plasma alpha-synuclein concentration is reduced in subjects with Parkinson's. It separates PD subjects from neurological controls with a statistical p value of P=0.04; and also separates the PD donors from those subjects that are healthy spouses of the PD donors. If reticulocyte count (P=0.0072) or the hematological blood cell factors (P=0.0056) are taken into account then the P value-based separation between PD and neurological controls is 10-fold higher.

Accordingly, the described method may also include a step of:
(f) quantifying the amount of total alpha-synuclein in plasma (e.g. by sandwich ELISA) and determining the complete blood count (e.g. by an established automated laboratory instrument such as by assessing value counts for erythrocytes, white blood cells, platelets, reticulocytes, hemoglobin (Hb), hematocrit (Hct), mean corpuscular volume (MCV), mean hemoglobin concentration (MHC), and mean cellular hemoglobin concentration (MCHC), where Hb, Hct, MCV, MCH, and MCHC represent the “hematological factor”) in a blood sample (e.g. a venous blood sample from a peripheral arm vein, preferably obtained at the same time the CSF sample was collected) obtained from said subject, and calculating a ratio of plasma alpha-synuclein to reticulocyte count and a ratio of alpha-synuclein to the aggregate hematological factor.

The neurodegenerative disease may be a alpha-synuclein related disorder such as sporadic Parkinson disease/Parkinsonism, familial Parkinson disease/Parkinsonism, sporadic or heritable Dementia with Lewy-Bodies, multiple system atrophy, Alzheimer's disease variants with Lewy body pathology, Down's syndrome variants with Lewy bodies, essential tremor with Lewy bodies, pure autonomic failure and neuropathy with alpha-synuclein deposition, incidental Lewy body disease associated with advanced age, lysosomal storage disorder with alpha-synuclein deposition, hereditary neurodegeneration with brain iron accumulation, familial Parkinson disease/Parkinsonism with dementia resulting from mutant genes, secondary Parkinson disease/Parkinsonism resulting from neurotoxin exposure/drug-induced Parkinsonism with alpha-synuclein deposition, Gaucher's disease with associated Parkinsonism, and conditions associated with central and/or peripheral alpha-synuclein accumulation in mammals. The rapid or severe neurodegenerative disease may alternatively be a disorder related to progressive neuronal damage such as Creutzfeldt-Jakob disease and other prion diseases, or an acute neurological disease such as an infectious illness of the brain (encephalitis, abscess), acute and subacute cerebral ischemia and hemorrhage (i.e., stroke, intracerebral and subarachnoid hemorrhages), and acute or subacute head trauma and spinal cord injury. In certain preferred embodiments, the neurodegenerative disease is Parkinson's disease (PD), dementia with Lewy bodies (DLB), multiple system atrophy (MSA), Alzheimer disease (AD), Creutzfeldt-Jakob disease (CJD), Bovine spongiform encephalopathy (BSE) or other prion disease.

The method may advantageously be used for early diagnosis of the neurodegenerative disease, and in certain embodiments may be employed using an enzyme-linked immunosorbent assay (ELISA) method to quantitate the amount of alpha-synuclein in the CSF sample. In such embodiments, one or more antibodies specific for mammalian alpha-synuclein (rodent, feline, canine, deer, bovine, equine, primate alpha-synuclein) will be employed, and colorimetrically-tagged, or luminescence-tagged, or fluorescence-tagged anti-alpha-synuclein antibody. In such cases the formation of an antigen-anti-alpha-synuclein antibody complex may be detected using enzyme-linked avidin. A non-limiting example of such a detection reagent would be ExtrAvidin alkaline phosphatase.

In an embodiment, the ELISA method is sandwich-based, and incorporates at least two (2) antibodies. Preferred embodiments of such antibodies incorporate the use two antibodies raised in different species. In this way the background of the assay can be further reduced.

When preparing the CSF sample, it may be preferred to incorporate a protease inhibitor or protease inhibitor (PI) cocktail, which can be added either after collecting the CSF (before freezing) or added after thawing it for the first time.

An advantage of one embodiment of the above-described method is that the CSF sample does not require pre-concentration. In such embodiments, the above-described ELISA method may be used together with a monoclonal alpha-synuclein antibody, for instance monoclonal anti-alpha-synuclein Ab commercially available from BD Transduction Labs.

According to the above-described method, a decrease in the ratio of alpha-synuclein to total protein in the CSF sample from the subject is typically correlated with a diagnosis of, or likelihood of developing Parkinson disease, multiple system atrophy, dementia with Lewy bodies or other alpha-synuclein related neurodegenerative disease. In certain preferred embodiments, such a diagnosis may be enhanced by:
(g) quantitating the amount of EDTA whole blood lysate alpha-synuclein and the complete blood count in a blood sample obtained from the subject (e.g. a venous blood sample obtained from the subject at the same time the CSF sample was collected) and calculating a ratio of EDTA whole blood lysate alpha-synuclein to reticulocyte count in the blood sample and of the ratio of alpha-synuclein to the aggregate hematological factor in the blood sample (see above), and
(h) comparing the ratio of alpha-synuclein to reticulocyte (or aggregate hematological factor) in the blood sample obtained from the subject with the alpha-synuclein to reticulocyte (or aggregate hematological factor) ratio in blood samples obtained from healthy subjects that at the time of sampling did not show clinical signs of neurodegenerative disease and that did not develop neurodegenerative disease; and
(i) comparing the ratio of alpha-synuclein to reticulocyte (or aggregate hematological factor) in the blood sample obtained from the subject with the alpha-synuclein to reticulocyte (or aggregate hematological factor) ratio in blood samples obtained from neurological subjects that at the time of sampling did not show clinical signs of a neurodegenerative disease linked to intracellular alpha-synuclein aggregation in the brain and that did not develop neurodegenerative disease linked to intracellular alpha-synuclein aggregation.

A reduction in the ratio of plasma alpha-synuclein to reticulocyte content in the blood sample from the subject (for instance by approximately >1.25 fold) indicates an increased correlation in the likelihood of developing Parkinson disease, dementia with Lewy bodies or other alpha-synuclein related neurodegenerative disease. The amount of plasma alpha-synuclein, total protein and complete blood count in the blood sample can be quantitated using a plasma and whole blood sample.

Similarly, a reduction in the ratio of plasma alpha-synuclein to aggregate hematological factor content in the blood sample from the subject (for instance by approximately >1.25 fold) indicates an increased correlation in the likelihood of developing Parkinson disease, dementia with Lewy Bodies or other alpha-synuclein related neurodegenerative disease. The amount of plasma alpha-synuclein, total protein and complete blood count in the blood sample can be quantitated using a plasma and whole blood sample.

A rise in the ratio of EDTA whole blood lysate alpha-synuclein to reticulocyte content in the blood sample from the subject (for instance by approximately >1.25 fold) indicates an increased correlation in the likelihood of developing Parkinson disease, dementia with Lewy bodies or other alpha-synuclein related neurodegenerative disease. The amount of EDTA whole blood lysate alpha-synuclein, total protein in the blood sample and the complete blood count can be quantitated using two whole blood samples.

A rise in the ratio of EDTA whole blood lysate alpha-synuclein to aggregate hematological factor content in the blood sample (for instance by approximately >1.25 fold) from the subject indicates an increased correlation in the likelihood of developing Parkinson disease, dementia with Lewy Bodies or other alpha-synuclein related neurodegenerative disease. The amount of EDTA whole blood lysate alpha-synuclein, total protein in the blood sample and the complete blood count can be quantitated using two whole blood samples.

In the methods described herein, CSF alpha-synuclein and the ratio, CSF alpha-synuclein to total CSF protein may also be used in the differentiation between degenerative cerebellar disorders linked to synucleinopathy (e.g. MSA-type C) versus other progressive cerebellar disorders.

Samples of whole blood, serum, plasma and EDTA whole blood lysate are collected and processed following known procedures in the field, and with standard tubes commercially available for serum collection and processing.

In alternate embodiments, an increase of alpha-synuclein concentration in the CSF sample (for instance by approximately >40 fold) may be correlated with a diagnosis of, or likelihood of developing a prion-related disease such as Creutzfeldt-Jakob disease.

Typically the subject will be a vertebrate, more typically a mammal. Human and bovine subjects, as well as sheep and deer are particularly envisioned. For instance, if the subject is a human the neurodegenerative disease might include Parkinson's disease (PD), dementia with Lewy bodies (DLB), multiple system atrophy (MSA), Alzheimer disease (AD) or Creutzfeldt-Jakob disease (CJD). Alternatively, if the subject is a bovine mammal the neurodegenerative disease might include Bovine Spongiform Encephalopathy (BSE) or other prion diseases.

Diagnostic kits and commercial packages incorporating the features of the above-described method are also encompassed within the present invention. Accordingly, the invention further relates to a kit for determining whether a subject has a likelihood to develop a neurodegenerative disease and/or for the diagnosis of a subject suffering from neurodegenerative disease, comprising an antibody that specifically recognizes alpha-synuclein, and instructions for quantitating the amount of total alpha-synuclein and total protein content in a cerebrospinal fluid (CSF) sample, plasma, serum and EDTA whole blood lysate obtained from the subject using the antibody and correlating a ratio of the total alpha-synuclein content to the total protein content with a likelihood of developing a neurodegenerative disease and/or a diagnosis of neurodegenerative disease.

The above-described kit and method may also be used in determining the rate of disease progression in a subject. For example, an annual decline in the alpha-synuclein concentration in the CSF and/or a decline in the ratio, CSF alpha-synuclein to CSF total protein concentration that exceeds the rate of change seen in neurologically and medically healthy individuals during the same observation period may serve as a progression marker of Parkinson disease (and related disorders). It is envisioned that such biochemical measures will be correlated with known clinical progression scores that encompass motoric (such as Hoehn and Yahr score; UPDRS score) and non-motoric scales, and that such a biochemical marker will be of value in clinical decision making and in clinical trials to determine the efficacy of pharmacotherapy.

In addition, measuring CSF synuclein in living animals such as deer and cattle can be used to prevent the slaughter usually required to diagnose prion diseases. Accordingly, the methods and kits described herein can be adapted for clinical subject monitoring purposes.

The kit will typically include an antibody such as those described above, e.g a biotinylated antibody specific for alpha-synuclein which can be detected using a detection reagent such as an enzyme-linked avidin. In select embodiments, the antibody will be biotinylated with twice the normal concentration of biotin, and the enzyme-linked avidin will be ExtrAvidin alkaline phosphatase.

The invention also relates to a method of testing a subject for a neurodegenerative disease or a predisposition for developing a neurodegenerative disease, characterized by the following steps:
quantifying the amount of total alpha-synuclein and of total protein in a cerebrospinal fluid (CSF) sample from said subject and calculating a ratio of total alpha-synuclein to total protein content;
comparing the ratio of total alpha-synuclein to total protein content in said CSF sample obtained from said subject with the alpha-synuclein to total protein content ratio in CSF reference samples obtained from neurologically healthy subjects from the same age range that at the time of sampling did not show clinical signs of neurodegenerative disease;
determining from the comparing step whether there is a difference in the ratio of total alpha-synuclein to total protein content in the CSF sample of said subject when compared with the ratio of alpha-synuclein to total protein content in the CSF reference samples obtained from the neurologically healthy subjects from the same age range; and outputting the results of said determining step.

In an embodiment, the above method may further comprise:
quantifying the amount of total tau protein in a cerebrospinal fluid CSF sample from said subject;
determining whether there is a rise in the content of total tau protein in the CSF sample of said subject when compared with the total tau level in the CSF samples obtained from neurologically healthy subjects from the same age range; and
outputting the results of said tau determining step.

In another embodiment, the above method may additionally comprise:
quantifying the amount of total alpha-synuclein in a sample of plasma and/or whole blood obtained from said subject,
determining the complete blood count,
calculating a ratio of plasma alpha-synuclein to reticulocyte count and a ratio of alpha-synuclein to the aggregate hematological factor, and/or a ratio of whole blood alpha-synuclein to reticulocyte count and a ratio of alpha-synuclein to the aggregate hematological factor;
determining whether there is a reduction in the ratio of plasma alpha-synuclein to reticulocyte content and plasma alpha-synuclein to aggregate hematological factor content in the sample from the subject; and/or a rise in the ratio of whole blood alpha-synuclein to reticulocyte content and whole blood alpha-synuclein to aggregate hematological factor content in the blood sample from the subject; and
outputting the results of said determining step.

The above method may also, in select embodiments, further comprise:
quantitating the amount of EDTA whole blood lysate alpha-synuclein in a blood sample obtained from the subject,
quantitating the complete blood count in a blood sample obtained from the subject,
calculating a ratio of EDTA whole blood lysate alpha-synuclein to reticulocyte count in the blood sample and/or a ratio of alpha-synuclein to the aggregate hematological factor in the blood sample;
comparing the ratio of alpha-synuclein to reticulocyte or aggregate hematological factor in the blood sample obtained from the subject with the alpha-synuclein to reticulocyte or aggregate hematological factor ratio in blood samples obtained from healthy subjects that at the time of sampling did not show clinical signs of neurodegenerative disease and that did not develop neurodegenerative disease; and
comparing the ratio of alpha-synuclein to reticulocyte or aggregate hematological factor in the blood sample obtained from the subject with the alpha-synuclein to reticulocyte, or aggregate hematological factor ratio in blood samples obtained from neurological subjects that at the time of sampling did not show clinical signs of a neurodegenerative disease linked to intracellular alpha synuclein aggregation in the brain and that did not develop neurodegenerative disease linked to intracellular alpha synuclein aggregation;
determining whether there is a reduction in the ratio of plasma alpha-synuclein to reticulocyte content and plasma alpha-synuclein to aggregate hematological factor content in the sample from the subject and/or a rise in the ratio of whole blood alpha-synuclein to reticulocyte content and whole blood alpha-synuclein; and
outputting the results of said determining step.

The invention also relates to a method of characterizing alpha-synuclein levels in a biological sample of a subject, characterized by steps of:
quantifying the amount of total alpha-synuclein in a cerebrospinal fluid (CSF) sample from said subject;
quantifying the amount of total protein in a CSF sample from said subject; and
outputting the results of said alpha-synuclein and total protein quantifying steps.

In an embodiment, the above method may also further comprise:
quantifying the amount of total tau protein in a CSF sample from said subject; and
outputting the results of said tau quantifying step.

In another embodiment, the above method may also comprise:
quantifying the amount of total alpha-synuclein in a sample of plasma and/or whole blood obtained from said subject,
quantifying reticulocyte count and/or aggregate hematological factor in a sample of plasma and/or whole blood obtained from said subject; and
outputting the results of said blood alpha-synuclein quantifying step and said reticulocyte count and/or aggregate hematological factor quantifying steps.

In yet another embodiment, the above method may further comprise:
quantifying the amount of EDTA whole blood lysate alpha-synuclein in a blood sample obtained from the subject;
quantifying reticulocyte count and/or aggregate hematological factor in a blood sample obtained from the subject; and
outputting the results of said EDTA whole blood lysate alpha-synuclein quantifying step and said reticulocyte count and/or aggregate hematological factor quantifying steps.

It should be understood that the above features of the above-described methods and kits can be interchangeably selected according to the usual knowledge of one skilled in the art.

Those skilled in the art will also recognize, or be able to ascertain using no more than routine experimentation, numerous equivalents to the specific methods, products and procedures described herein. Such equivalents are considered to be within the scope of this invention and are covered by the appended claims.

### BRIEF DESCRIPTION OF THE FIGURES

Further details of the invention will become apparent from the detailed description, taken in combination with the following appended figures:

FIG. 1. Purification (a) and trypsin digest map (b) of alpha-synuclein. (a) Affinity-enrichment of alpha-synuclein (αS) from mouse brain (upper panel) and from cell-free, human cerebrospinal fluid (lower panel). Upper panel: Western blotting of starting material (start) from mouse brain homogenate, eluates #1 to #7, and flow through, as stained with mAb, Syn-1; Lower panel: Western blotting of starting material from cell-free, human CSF, eluates #1 to #7, and its flow through, as stained with mAb, Syn-1; (b) Wild-type sequence of the human α-synuclein protein (SEQ ID NO. 1), including map of the 16 predicted, trypsin (T) digestion-generated fragments of full-length, human α-synuclein. The identified tryptic fragments of CSF alpha-synuclein identified are listed in Table 1 (below).

FIG. 2. Development of second-generation ELISAs for recombinant α-synuclein quantification. (a) Serial dilutions of recombinant human αS, of mouse wild-type brain and snca-null extracts, and of unconcentrated normal human CSF were loaded in duplicate onto 384-well plates and read by ELISA using variable antibody combinations, as indicated at the top. For sandwich [mSA-1/Syn1-BB], samples were loaded onto a separate 384-well plate and developed in parallel according to longer incubation periods. Asterisk denotes no signal (after subtraction of blank values). Note, 211 is a human αS protein-specific monoclonal antibody. (b) Serial aliquots of recombinant, human αS were loaded in triplicate onto 3 different segments of a 384-well plate in 3 runs separated by 3 days each and developed by ELISA [mSA-1/Syn1-B]. The day-to-day inter-assay variability is depicted by shaded bars.

FIG. 3. Characterization of ELISA protocol and optimization for detection of CSF α-synuclein. (a) Quantification of αS in native CSF from two separate donors (CSF-I and -II), and in CSF spiked with aliquots of recombinant human αS, as analyzed in duplicate by ELISA [mSA-1/Syn1-BB] on a 384-well plate. The recovery rate of spiked recombinant protein was calculated and listed above. The absorbance signal curve for the recombinant protein is shown below. (b) Identical aliquots of cell-free, native, previously unfrozen CSF from a single donor were processed in parallel under six different conditions, as indicated. Samples were loaded in duplicate onto a 384-well plate and developed by ELISA [mSA-1/Syn1-BB]. +PI denotes the addition of protease inhibitors; NP40, presence of 0.5% detergent; ‘non-silicon.’, use of non-siliconized tubes. The detection rate (in percent) is calculated for each experimental arm with the +PI+NP40 aliquot assigned as 100.

FIG. 4. Cross-sectional examination of CSF α-synuclein by ELISA. Aliquots of unconcentrated CSF from 80 living subjects were loaded in duplicate onto 384-well plates and analyzed by ELISA [mSA-1/Syn1-BB]. Five diagnostic groups were examined: AD denotes Alzheimer disease; PD, Parkinson disease; DLB, dementia with Lewy bodies; NCO, neurological controls; and CJD, Creutzfeldt-Jakob disease. Note, CSF specimens from CJD cases were diluted (1:40) prior to loading. The value for a single patient with autopsy confirmation is marked in the DLB cohort. (□ mean; ▪ mean±standard error of the mean; I bars indicate mean±0.9 confidence interval). The demographic details for these CSF donors is listed in Table 2.

FIG. 5. Post mortem examination in index patient with DLB confirms synucleinopathy. (a) Formalin-fixed, paraffin-embedded brain sections from a subject clinically diagnosed with probable dementia with Lewy bodies (DLB). At autopsy, representative sections of frontal lobe cortex (CTX), locus coeruleus (LC) and substantia nigra (SN) were probed for αS reactivity using anti-αS Ab, LB509. The clinical diagnosis of DLB was confirmed by the presence of cortical and brainstem Lewy body (LB) pathology. Regular arrows identify LB-carrying neurons; open arrow depicts a neuromelanin-carrying neuron without LB. (b) Analysis of CSF αS concentration collected from one living control donor and one living (and subsequently autopsy-confirmed) dementia with Lewy body (DLB) patient (from (a)), and of CSF and frontal cortex collected post mortem from the same DLB subject. The αS concentrations were measured in duplicates using ELISA [mSA-1/Syn1-BB] on a 384-well plate. Note, the low CSF αS concentration that is detected before death in the DLB patient (vs. control), and the >300-fold elevation in CSF αS at 12 h following brain death. The logarithmic graph also highlights the low picogram (per μl) amounts of αS that are detectable in adult human CSF when compared with the soluble αS concentrations (in nanograms/μl) recorded in brain tissue from the same donor.

FIG. 6. Detection of α-synuclein in the medium of healthy neurons. (a) Rat primary cortical neurons transduced with IRES-GFP or SNCA-IRES-GFP lentiviruses (LV) were fixed at day-in-vitro (DIV) 8 and immunostained against either αS or GFP. Cells transduced with SNCA-IRES-GFP lentivirus exhibited stronger staining than those transduced with IRES-GFP lentivirus. Scale bar, 50 μm. (b) Detection of αS in the conditioned medium (CM) of transduced neurons. The CM of untransduced neurons or those transduced with either IRES-GFP or SNCA-IRES-GFP lentiviruses were harvested at DIV 8 and analysed for αS content by ELISA [hSA-2/Syn1-13]. Results show the mean (±SD) concentration measurements (in pg/μl) of CM from duplicate dishes of neurons. (c) Time course of appearance of endogenous αS in the CM of rat primary cortical neurons plated on embryonic day 18. CM was sampled over nine days and assayed for their αS content by ELISA [hSA-2/Syn1-B]. Results show the mean (±SD) concentration measurements in pg/gl.

FIG. 7. Independent cross-sectional studies of neurological subjects (Caucasian) with probable diagnoses (A-D) and definite diagnoses (E). Aliquots of previously frozen cerebrospinal fluid (CSF) from 273 living, age-matched donors collected by lumbar puncture were analyzed by ELISA (mSA1/Syn1-BB). Box plot graphs are shown for CSF α-synuclein concentrations (A) and the ratio of α-synuclein-to-total-protein content in CSF (B). Five diagnostic groups were examined: AD denotes Alzheimer disease; PD, Parkinson disease; DLB, dementia with Lewy bodies; NCO, neurological controls; and MSA, multiple system atrophy. Additional aliquots of previously frozen cerebrospinal fluid (CSF) from 109 of the 273 living donors (that were shown in A and B) were analyzed by ELISA measuring the total tau concentration in CSF. Box plot graphs are shown for CSF tau concentrations (C). Graph in D demonstrates the area under the curve value (AUC=c) in regression analyses of donors shown in FIG. 7A-C. AUC values (≦1.0) determine the specificity and sensitivity of the probability to correctly distinguish between being MSA vs non-MSA cases (c=0.794); PD versus non PD cases (c=0.878); and of synucleinopathy (PD, MSA, DLB; c=0.908) versus non-synucleinopathy cases (AD, NCO) based on the following parameters: age, total CSF protein, total CSF alpha-synuclein, and total CSF tau. (E) Aliquots of previously frozen cerebrospinal fluid (CSF) from 41 living and subsequently deceased, autopsy confirmed donors collected by lumbar puncture were analyzed by ELISA (mSA1/Syn1-BB). Box plot graphs are shown for CSF α-synuclein concentrations (E). Three definite diagnostic groups were examined: AD denotes Alzheimer disease; DLB, dementia with Lewy bodies; NDC, neurological disease controls. The latter carried a definite diagnosis of autopsy-proven corticobasal degeneration, cerebrovascular disease, subacute atherosclerotic encephalopathy, and tau-positive frontotemporal dementia. In addition, in three of the NDC cases the diagnosis of Huntington disease was established by DNA analysis.

FIG. 8. Concentration of α-synuclein in biological fluids, including in EDTA whole blood lysates, plasma, serum, CSF from healthy donors, and in cell lysates over-expressing human SNCA. Note, log-transformed scale for absolute alpha-synuclein concentration.

FIG. 9. Representative plasma levels of α-synuclein: Whisker Plots showing that mean plasma levels are reduced in patients with Parkinson's (PD) versus healthy controls and neurological controls (A) at the time of enrollment in 318 donors and (B) at the time of a 1-year follow up return visit (80 donors examined).

FIG. 10. Statistical analysis of mean total plasma α-synuclein values in a cohort of 318 donors [50 percent PD; 25 percent healthy control (HC) individuals; 25 percent neurological control (NC) subjects] showing 15 readings by ELISA in 2 minute intervals comparing the three diagnostic groups (triangle, PD; square, NC; circled line, HC). (A) ANOVA-based comparison of raw values; (B) ANCOVA covarying total protein; (C) ANCOVA covarying age; (D) ANCOVA covarying processing time after phlebotomy; (E) ANCOVA covarying reticulocyte count; and (F) ANCOVA covarying aggregate hematological factor.

### DETAILED DESCRIPTION

The present inventors have examined the occurrence of alpha-synuclein (also referred to herein as alpha-synuclein and alpha-synuclein) in human cerebrospinal fluid (CSF) and, following affinity enrichment and trypsin digestion of CSF collected from a neurologically healthy donor, were able to identify several alpha-synuclein-derived peptides by mass spectrometry. The concentration of alpha-synuclein amounted to <0.001% of the CSF proteome.

Table 1a lists the currently known human conditions that are associated with microscopic evidence of alpha-synuclein mishandling. These intracellular alpha-synuclein-related pathologies cannot yet be visualized in living subjects without brain biopsy; hence the need for a convenient assay to allow for monitoring and measuring alpha-synuclein-associated abnormalities.

TABLE 1a
Neurological syndromes with microscopic
evidence of synucleinopathy

A. Invariable synucleinopathy of neurons in:
Parkinsonism
Sporadic Parkinson disease
Familial, SNCA-linked Parkinson disease
Dementia
Sporadic, pure dementia with Lewy bodies
Familial, SNCA-linked dementia with Lewy bodies
Lewy body variant of Alzheimer disease
Familial, APP-linked Alzheimer disease
Down syndrome
Familial, Presenilin1-linked Alzheimer disease
Familial, Presenilin2-linked Alzheimer disease
Neuroaxonal dystrophy
Sporadic neurodegeneration with brain iron accumulation (i.e.,
Hallervorden-Spatz disease)
Familial, PANK2-linked neurodegeneration with brain iron accumulation
Lysosomal storage disease
Familial, CTSD-linked neuronal ceroid lipofuscinosis
Pure autonomic failure
Incidental Lewy body disease (in the absence of known neurological
deficits)
B. Invariable synucleinopathy of glia in:
Multiple system atrophy
Parkinsonism variant (i.e., striatonigral degeneration)
Ataxia variant (i.e., olivopontocerebellar degeneration)
Autonomic variant (i.e., Shy-Drager syndrome)
C. Variable synucleinopathy in:
Parkinsonism
Familial, LRRK2-linked Parkinson disease
Familial, Parkin-linked Parkinson disease
Dementia with parkinsonism
Pick disease
Progressive supranuclear palsy (i.e., Steele-Richardson syndrome)
Prion disease (e.g., Creutzfeldt-Jakob disease)
Neurovisceral storage disease
Familial, GBA-linked Gaucher disease
Familial, NP1C-linked Niemann-Pick disease
Other condition
Juvenile onset neuroaxonal dystrophy
Amyotrophic lateral sclerosis
Traumatic brain injury

A sandwich-type, enzyme-linked immunoadsorbent assay (ELISA) was then developed, validated and optimized to measure total alpha-synuclein levels in unconcentrated CSF. Several generations of the assay have been developed as described in further detail below, the third generation having been optimized to a 384-well ELISA format using un-concentrated CSF samples. However, it should be understood that the assay methodology and diagnostic kits of the invention can be adapted in a variety of ways according to the usual knowledge and skill of one in the art. For instance, the assay may be carried out in alternate plate formats readily available, such as 48-, 96-, 384- or 1536-well plates, or in individual cuvettes. For instance, chemiluminescence and fluorescence are envisioned as non-limiting alternatives to the biotin—streptavidin and alkaline phosphatase read-out method employed in the experiments. Further, the invention may be practiced in multiplex systems, e.g. where several proteins are measured in the same sample at the same time.

The term “antibody” as used herein encompasses functional fragments of antibodies, including fragments of chimeric, humanized, primatized, veneered or single chain antibodies. Functional fragments include antigen-binding fragments of antibodies that bind to alpha-synuclein polypeptide or a portion thereof. For example, antibody fragments capable of binding to an alpha-synuclein polypeptide or a portion thereof include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

Antibodies specifically recognizing alpha-synuclein may include monoclonal (e.g. mouse and/or rabbit-based) or polyclonal (e.g. rabbit and/or goat-based) antibodies as may be prepared according to known methods and with appropriate mammalian species. Also, other antibodies commercially available or described in the art that recognize alpha-synuclein can be used.

The monoclonal antibodies used in a method of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies used in a method of the invention may be human monoclonal antibodies. The term “humanized antibody” means that at least a portion of the framework regions of an immunoglobulin is derived from human immunoglobulin sequences.

The antibodies used in a method of the present invention may further be labeled by an appropriate label of the enzymatic, fluorescent, or radioactive type.

In addition, it is envisioned that the anti-αS antibodies used in the method can be in an immobilized state on a suitable support (for instance in a microtiter plate well). Further, the immunological binding of the anti-αS antibodies may be detected by a specific marker. Advantageously, a secondary antibody itself carrying a marker or a group for direct or indirect coupling with a marker will be employed for detection.

Alternatively, the method may be put into practice by using any other immunoassay format known to the person skilled in the art, or combined with detection and/or quantitation methods involving other neurological markers. For instance, quantitation of αS in CSF as described herein may be combined with a method for detecting one or more other proteins associated with certain types of neuronal cells or cell function, of which the level in one or more body fluids under conditions of neurodegeneration is indicative for the disease process or the cause of neurological disorder. Some neurological markers may be elevated and others reduced in one or more body fluids under a certain neurological condition. Any possible combination of 3, 4, 5, 6, 7, 8 or more neurological markers that have an altered level in a certain body fluid under a certain neurological condition can be used for the specific detection, quantification and/or differential diagnosis of said neurological condition in an individual. Possible neurological markers might include: total protein concentration, tau, neuron-specific enolase (NSE), beta-amyloid, amyloid precursor protein (APP), APP-like proteins, neuromodulin, synapse proteins (such as Rab3a, SNAP25, synapsin, synaptotagmin, synaptobrevin, syntaxin, rabphilin, cysteine string protein and others), glial fibrillary acidic protein (GFAP), SIOO, EL6, TNF, IL1, IL2, neurofilament (NF), myelin basic protein (MBP), 14-3-3, Parkin, cathepsin proteins, ubiquitin, homovanilinic acid, dopamine, dopamine quinine, and metanephrin. However, this list is not complete. Other neurological markers that are indicative for a certain disease process or cause of neurological disorder can be used as well.

In a cross-sectional study of 100 living donors, cell-free CSF samples from subjects clinically diagnosed with advanced PD, dementia with Lewy bodies (DLB), Alzheimer disease (AD), and a group of non-neurodegenerative disease controls (NCO) were examined. In these four groups the CSF αS concentrations ranged from 0.8 to 16.2 pg/μl. Mean CSF αS values were lower in donors with a primary synucleinopathy (PD, DLB: n=57) than in the other two groups (AD, NCO: n=35; p=0.025). By contrast, living Creutzfeldt-Jakob disease patients showed markedly elevated CSF αS levels (n=8; mean, 300 pg/μl; p<0.001). These results confirm the presence of αS in adult human CSF and establish a link between relatively low CSF αS concentration in subjects with parkinsonism to synucleinopathy, PD and DLB. These data were confirmed in a second and third cohort analysis comprising 273 and 41 donors, respectively. In prion disease cases, on the other hand, a marked rise in total CSF αS was observed resulting from rapid cell death.

The invention accordingly provides an ex vivo method of diagnosing neurodegenerative diseases, including Parkinson disease, Creutzfeldt-Jakob disease and other α-synuclein and/or neuronal cell loss-related diseases (such as MSA and BSE). Moreover, the ELISA method which was employed displayed good sensitivity and specificity so that it particularly enables discriminating of these diseases.

The ex vivo method of diagnosing neurodegenerative diseases is particularly suitable for assisting in the making of early stage diagnoses. Such a method will be useful for diagnosticians and in clinical trials for making a diagnosis of disease state. This is particularly important since, e.g. in clinical trials, there is a currently a reliance on expensive, not widely available and inaccurate imaging technologies, or on biomarkers that lack specificity and sensitivity. The method is also useful in the food and veterinary industries to facilitate prion disease testing e.g. for BSE (mad cow disease), where the marker 14-3-3 is currently used as well as post-mortem immunohistochemistry to make a diagnosis of the disease state.

The ex vivo method of diagnosing neurodegenerative diseases is also suitable for assisting in the assessment of disease progression. Such a method will be useful for diagnosticians and in clinical trials for making a diagnosis of disease rate and response to pharmacotherapy (neuroprotection). This is particularly important since, e.g. in clinical trials, there is a currently a reliance on expensive, not widely available and inaccurate imaging technologies, or on biomarkers that lack specificity and sensitivity, or on rating scales that are not sensitive enough to detect change over a period of less than 6 months.

The ex vivo method of diagnosing neurodegenerative diseases is also suitable for assisting in the assessment of disease risk. Such a method will be useful for diagnosticians and in clinical trials for making a diagnosis of disease risk (before its clinical expression) and delaying its manifestation as a result of pharmacotherapy (neuroprevention). This is particularly important since, e.g. in clinical trials, there is a currently a reliance on expensive, not widely available and inaccurate imaging technologies, or on biomarkers that lack specificity and sensitivity.

In certain embodiments, the ELISA assay will be optimized for the direct quantification of alphaS in a predetermined amount of unconcentrated cerebrospinal fluid, e.g. a 50 μl sample for a 386-well plate-based assay format, or a 100 μl sample for a 96-well plate-based assay format. A non-limiting, sample protocol for carrying out the assay using a 50 μl CSF sample is provided below:

### ELISA Protocol:

Day 1-3 (can be Shortened to 36 Hours)
1. ELISA plate is coated with mSA2 (or hSA4) antibody [e.g. affinity purified anti-alpha-synuclein antibodies] diluted (1:300) in 50 μl per well in 200 mM NaHCO₃ with 0.01% sodium azide buffer, pH 9.6.
2. Plate is incubated at 4° C. for 42 hours (or up to 36 hours) on a shaker.

Day 4
1. ELISA plate is washed once with PBST (0.025% Tween-20).
2. 50 μl of blocking buffer [1.25 ml of 45% gelatin from Cold Water Fish Skin and 12.5ul Tween-20 (0.025%) in 50 ml PBS] are added to each well.
3. Plate is incubated at 37° C. for 2 hours.
4. After 2 hours, plate is washed 3 times with PBST .
5. 50 μl of samples are loaded into each well (in triplicates).
6. Plate is incubated at 4° C. overnight on a shaker.

Day 5
1. ELISA plate is washed once with PBST.
2. 500 of double biotinylated Syn1 antibody (Syn1-BB) are added to each well at 1:2000 dilution in blocking buffer and incubated at 37° C. for 3 hours.
3. Plate is washed 3 times with PBST.
4. 50 μl of ExtrAvidin Alkaline Phosphatase are added to each well by diluting 6 μl ExtrAvidin in 5 ml blocking buffer and incubated at 37° C. for 1.5 hours.
5. Plate is washed 3 times with PBST.
6. 50 μl of light sensitive Sigma Fast p-Nitrophenyl Phosphate are added to each well by diluting 1 tablet of Tris buffer and 1 tablet of pNPP buffer in 5 ml ddH₂O.
7. Plate is read @405 nm every 2.5 minutes (150 secs) for 1 hour immediately.

### CSF Sample Processing

1. 4.25 μl of 16% NP40/Protease Inhibitor mix (final 0.2% NP40/PI mix) are added to 340 ul of CSF sample (source: either freshly centrifuged following lumbar puncture or frozen at −80° C.).
2. The microtube containing sample is vortexed well and spun at 15,000 g for 10 mins at 4° C.
3. 20 μl sample are transferred into a new microtube and mixed well before loading onto an ELISA plate.

### Biotinylation of Syn1 Antibody

1. 400 μg of Pierce Sulfo-NHS-LC Biotin are added to 1 ml of BD Transduction Syn1 antibody.
2. Antibody-biotin mix is incubated on ice for 2 hours with gentle mixing every 10 mins.
3. Antibody-biotin mix is transferred into Biorad BioSpin 6 Chromatography Column and spun following Biorad protocol.
4. Flow-through is collected.

As mentioned above, there is also provided within the instant disclosure a test kit for carrying out the disclosed method. Such test kits can comprise, e.g., an antibody or antigen-binding fragment of the invention and one or more ancillary reagents suitable for detecting the presence of a complex between the antibody or antigen-binding fragment and an alpha-synuclein polypeptide or portion thereof. The antibody and antigen-binding fragments can be provided in lyophilized form, either alone or in combination with additional antibodies specific for other epitopes. The antibodies or antigen-binding fragments thereof, which can be labeled or unlabeled, can be included in the kits with adjunct ingredients (e.g., buffers, such as Tris (Tris(hydroxymethyl)aminomethane), phosphate and carbonate, stabilizers, excipients, biocides and/or inert proteins, e.g., bovine serum albumin). For example, the antibodies or antigen-binding fragments can be provided as a lyophilized mixture with the adjunct ingredients, or the adjunct ingredients can be separately provided for combination by the user. Generally these adjunct materials will be present in less than about 5% by weight based on the amount of active antibody, and usually will be present in a total amount of at least about 0.001% by weight based on antibody concentration. Where a second antibody or antigen-binding fragment capable of binding to the anti-alpha-synuclein antibody or antigen-binding fragment is employed, such antibody or fragment can be provided in the kit, for instance in a separate vial or container. The second antibody or antigen-binding fragment, if present, is typically labeled, and can be formulated in an analogous manner with the antibody formulations described above. The antibodies, antigen-binding fragments and/or ancillary reagent of the kit can be packaged separately or together within suitable containment means (e.g., bottle, box, envelope, tube). Additional tubes, vials, syringes, etc. can also be included within the kit for collection and processing of biological samples as appropriate. When the kit comprises a plurality of individually packaged components, the individual packages can be contained within a single larger containment means (e.g., bottle, box, envelope, tube).

As already noted, the above assay methodology can be adapted in a variety of ways according to the usual knowledge and skill of one in the art. Further embodiments may also be envisioned having regard to the following Examples, and such variations and embodiments are intended to be included within the scope of the invention as defined by the appended claims.

### EXAMPLES

### 1. Direct Quantification of CSF α-Synuclein by ELISA and Cross-Sectional Study in Patients With Neurodegeneration

### Materials and Methods:

### Study Participants

CSF donors presented with a variety of neurological conditions for admission to the Departments of Neurology and Psychiatry at the University of Goettingen and the nearby Paracelsus-Elena Klinik (Germany). Samples from CJD patients were collected at the ‘National Surveillance Unit for Spongiform Encephalopathies’ in Goettingen under the same conditions. The study was approved by the ethics committees at the University of Goettingen, the board of registration in Hessen, Germany, and at Brigham and Women's Hospital. CSF collection was carried out according to the Declaration of Helsinki with the informed consent of all patients or their next of kin in the case of dementia. The specimens from 100 living donors were collected by routine lumbar puncture (LP) into serial polypropylene tubes, as described (Andreasen et al., 1999, Lewczuk et al., 2006, Mollenhauer et al., 2006c and Otto et al., 2002). CSF (>40 ml) was also obtained by lumbar drain from an additional control donor, who had been treated for a persistent CSF leak in the absence of neurological deficits. Each CSF donor underwent neuroimaging to search for a structural cause of illness. The clinical diagnosis of ‘definite PD’ was rendered according to the UK Parkinson's Disease Society Brain Bank criteria (Gibb, 1988). DLB patients had dementia before the onset of parkinsonism and fulfilled both DSMIV criteria for dementia and McKeith criteria for the diagnosis of ‘probable DLB’ (McKeith et al., 2005 and McKeith et al., 1996); AD patients fulfilled DSMIV criteria for AD, and NINCDS-ADRDA criteria for the clinical diagnosis of ‘probable AD’ (McKhann et al., 1984). All medical charts were reviewed by a neurologist with subspecialty training in movement disorders and dementia (B.M.). For recruitment into our study, we first randomly surveyed frozen CSF aliquots, and then selected those specimens with a volume of >500 μl. In addition, ventricular CSF samples were collected during autopsy at the Department of Pathology of Columbia University (NY.) and in the case of one DLB patient at the Department of Neuropathology at the University of Goettingen. Autopsy consent was provided by the next of kin and CSF was obtained in accordance with both institutions' ethics guidelines.

All CSF specimens were centrifuged at 3200 rpm at 4° C. (10 min) within 30 min of their collection and stored at 4° C. until routine diagnostics were completed (including cell counts). Aliquots were then stored at −80° C. until ELISA preparation. Upon thawing on ice, NP40 (0.5%) and EDTA-free protease inhibitors (Roche, Germany) were added and samples transferred into siliconized Eppendorf tubes made of polypropylene. Centrifugation followed at 15,000×g at 4° C. for 10 min. The total protein concentration of each CSF specimen was measured independently by the method of Bradford (Bio-Rad).

### Affinity Enrichment and Mass Spectrometry

A non-alpha-synuclein directed immunoglobulin column (7071FT-Ig) and an alpha-synuclein-specific, polyclonal antibody (Ab; mSA-1) column were prepared using 50 mM sodium borate, pH 8.2, and 1 ml immobilized Protein G (ImmunoPure, Pierce) as per manufacturer's instructions. Mouse brain homogenates (Mollenhauer and Schlossmacher, 2008) and subsequently, CSF, were first applied onto the 7071FT-Ig column to remove non-specifically bound proteins. The flow through of the 7071 FT-Ig column was then applied to the mSA-1 Ab column. The latter was extensively washed and proteins were eluted with 3.5 ml elution buffer (0.2 M glycine, pH 2.0) into 7 fractions to collect alpha-synuclein and alpha-synuclein-associated proteins. The pH of eluates was equilibrated with 1 M Tris(pH 7.5). Eluates #2 and #3 from mouse brain and human CSF were reduced in 8 M urea/1% SDS/100 mM ammonium bicarbonate/10 mM DTT (pH 8.6), before being alkylated in 30 mM iodoacetamide. Each eluate was re-electrophoresed on an 8-16% Tris/glycine gel, stained with Simply Blue Safe Stain (Invitrogen Life Technologies), imaged and sliced horizontally. Gel slices were destained, washed, rinsed and dried before being digested with sequencing-grade trypsin (Promega) for 36 h. Peptides were extracted, vacuum concentrated and loaded onto a 75 mm nanospray capillary linked to a LCQ DECA XP plus mass spectrometry (MS) instrument (Thermofinnigan). Each LCQ MS scan was followed by three MS/MS scans. Peptide identifications were made using Sequest (Bioworks Browser version 3.2), and database searches were performed using the RefSeqHuman FASTA database from the EMBL European Bioinformatics Institute.

For validation purposes, we first examined this protocol's ability to enrich alpha-synuclein from adult mouse brain. Two fractions of the newly built affinity column, which contained high levels of murine alpha-synuclein by SDS/PAGE/Western blotting were further prepared for trypsin digestion and MS. Among the 177 peptides identified in fractions #2 and #3 from mouse brain, 26 fragments corresponded to four murine alpha-synuclein-specific sequences. Note, these column eluates did not contain any human peptide sequence, as expected (Mollenhauer and Schlossmacher, 2008).

### Development of Second-Generation ELISAs

Sera from six rabbits were raised and subsequently affinity-purified at Open Biosystems against bacterially expressed full-length alpha-synuclein of the wild-type human (hSA-2; 7071AP) and mouse (mSA-1) variant. Antigens were prepared as previously described (Shtilerman et al., 2002); their purity was independently assessed by HPLC, amino acid composition analysis and MS. Monoclonal Ab Syn-1 was from BD Transduction Labs.

For second-generation ELISAs, 96- and 384-well MaxiSorp plates (Nunc, Inc) were coated with capturing purified Ab diluted in coating buffer (NaHCO₃ with 0.2% NaN₃, pH 9.6) in 200 μl and 50 μl volumes/well, respectively. Following washes with PBS/0.05% Tween-20 (PBS-T), plates were blocked for 2 h at 37° C. in blocking buffer (1.125% fish skin gelatine; PBS-T). After 4 washes, samples were loaded and incubated at 4° C. for 12 h. Biotinylated Syn-1 mAb (as the assaying Ab) was generated using 200 μg Sulfo-NHS-LC Biotin (Pierce), diluted in blocking buffer and added to the plate for 2 h at 37° C. Following 4 washes, ExtrAvidin phosphatase (Sigma) diluted in blocking buffer was applied for 1 h at 37° C. Color development was carried out by using fast-p-nitrophenyl phosphate (Sigma) and monitored at 405 nm every 5 min for up to 60 min. Saturation kinetics were examined for identification of time point(s) where standards and sample dilutions were in the log phase. The [mSA-1/Syn1-BB] protocol is a modification of ELISA [mSA-2/Syn1-B] for the direct measurement of alpha-synuclein in native CSF. Plates were coated for 48 h at 4° C. with mSA-1. Duplicate (or triplicate) samples of cell-free CSF were incubated for 48 h at 4° C. After PBS-T washes, Syn-1 Ab biotinylated with twice the normal concentration of biotin (BB; 400 μg/ml biotin; dilution, 1:2000) was applied. After four washes, ExtrAvidin alkaline phosphatase was added (dilution, 1:800; 1.5 h). A standard series of recombinant, full-length human alpha-synuclein diluted in blocking buffer was run in parallel. Each plate contained ‘blank wells’ (PBS/1× protease inhibitors/0.5% NP40) and as an internal standard, aliquots of two known, previously prepared CSF specimens. Aliquots of frozen adult human and mouse brain tissues were homogenized and processed, as described (Schlossmacher and Shimura, 2005).

### Primary Neuronal Cultures

Whole brains were dissected from E18 rats, and the cortices were mechanically dissociated in neurobasal medium (NBM) containing 10% fetal bovine serum. Neurons were plated onto 35 mm dishes or 4-chamber glass slides; 1.5 h after plating the medium was replaced with NBM supplemented with B27. Neurons were transduced at day 5 in vitro by lentivirus encoding IRES-GFP or SCNA-IRES-GFP.

After a 72 h period, the conditioned media (CM) were harvested. A portion of CM was analyzed for adenylate kinase activity as a measure of cell viability, using the ToxiLight™ kit (Cambrex) according to the manufacturer's protocol. To examine GPF and alpha-synuclein expression, monolayers were washed, fixed with 4% paraformaldehyde and permeabilized in 0.1% Triton X-100, and blocked with 1.5% normal goat serum. Staining was performed with mouse anti-alpha-synuclein Syn-1 and rabbit anti-GFP (Abeam) and visualized using goat anti-mouse IgG-Cy3 1:500 (Rockland Immunochem) and goat anti-rabbit IgG-Alexa488, 1:300 (Molecular Probes). CM was harvested, EDTA-free protease inhibitors (Roche) added and cleared at 100,000 g (30 min) at 4° C.; the top ⅔ of supernatants were removed and frozen until assay.

### Immunohistochemistry

The alpha-synuclein reactivity of brain was visualized by routine immunohistochemistry, as published (Schlossmacher and Shimura, 2005). For neuropathological confirmation of the DLB index case and immunohistochemistry sections, staining was carried out using monoclonal anti-alpha-synuclein Abs, Syn-1 and LB509 (Zymed) (Kramer and Schulz-Schaeffer, 2007).

### Statistical Analysis

All analyses were performed by a statistician using T-tests, ANOVAs, ANCOVAs with post hoc tests, and logistic regression analysis, as indicated in the text. Monte Carlo approximated permutation tests, Satterthwaite T-tests, or log transformed dependent variables were employed whenever assumptions (normality, homogenous variances) of conventional tests appeared to be violated. In the case of significant differences in age and CSF total protein content among different groups, a covariant adjustment was carried out, as indicated in the text.

Detection of alpha-synuclein in human CSF by mass spectrometry

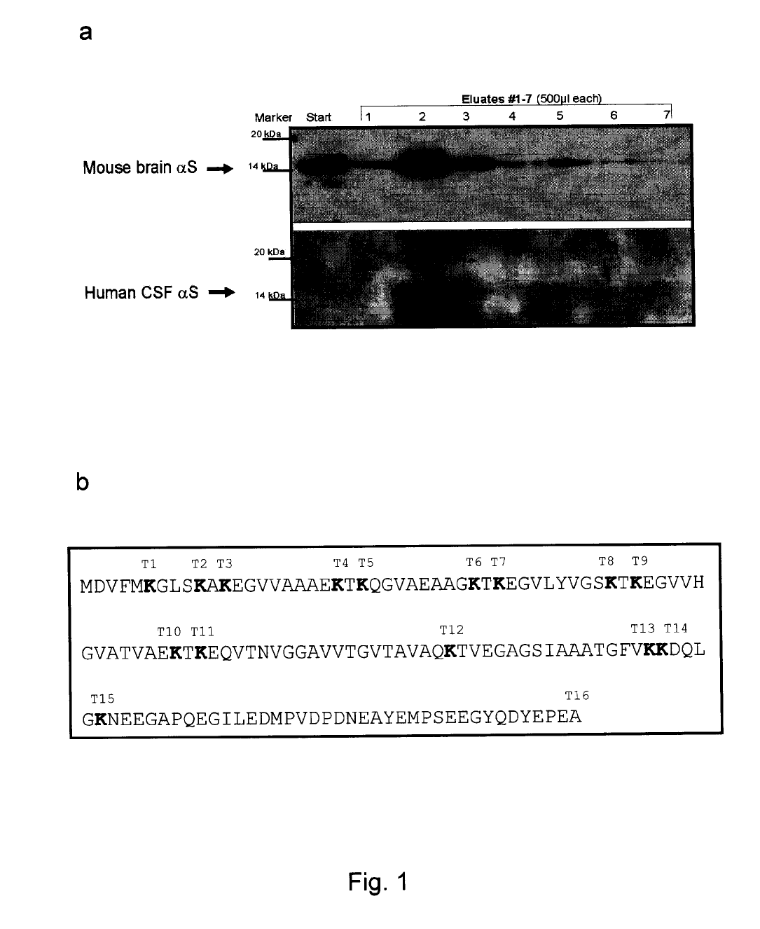

To collect structural evidence in support of the presence of alpha-synuclein in cell-free CSF, we first built and validated a protocol for its enrichment from mammalian brain (Materials and methods) (Mollenhauer and Schlossmacher, 2008). We then collected human CSF from a neurologically intact subject, who carried an indwelling lumbar drain (LD-CSF) because of a leak. We loaded 40 ml of LD-CSF onto our serial Ab column system that heretofore had never been exposed to a human alpha-synuclein-containing specimen. Eluates #2 and #3 of the anti-mouse alpha-synuclein Ab (mSA-1) affinity column, which contained the 16 kDa, full-length alpha-synuclein reactivity (FIG. 1a), were then prepared for mass spectrometry (MS). As shown in Table 1, MS revealed 10 of the 16 predicted fragments of full-length alpha-synuclein, which corresponded to a total of 76 residues of the 140 amino acid-long protein (FIG. 1b). Importantly, with residues Ala 53 and Ser 87 in fragments #10 and #13, respectively, the identity of the LD-CSF-derived protein as the human alpha-synuclein orthologue was unequivocally confirmed (Table 1).

TABLE 1
Human alpha-synuclein peptides identified in cerebrospinal fluid by mass spectrometry.

| Human CSF | | | | | | | Peptide |
|---|---|---|---|---|---|---|---|
| Trypsin-generated | Peptide fragment sequence | | | Score | MW | Accession | (Hits) |
| peptide fragment # | Column eluate #2 | MH+ | Sf | XC | Sp | RSp | Ions |
| | Alpha-synuclein isoform | | 4.75 | 50.31 | 14,460.1 | 4507109 | (200000) |
| | NACP140 | | | | | | |
| T3-T4 | K.AKEGVVAAAEK.T | 1072.59970 | 0.56 | 3.06 | 185.2 | 1 | 14/30 |
| T9-T10 | K.TKEGVVHGVATVAEK.T | 1525.73191 | 0.93 | 3.95 | 1136.5 | 1 | 21/42 |
| T10 | K.EGVVHGVATVAEK.T | 1296.45466 | 0.94 | 3.70 | 1044.6 | 1 | 24.36 |
| T12 | K.EQVTNVGGAVVTGVTAVAQK.T | 1929.16446 | 0.95 | 4.63 | 912.3 | 1 | 23/57 |
| T11-T12 | K.TKEQVTNVGGAVVTGVTAVAQK.T | 2158.44170 | 0.98 | 6.29 | 1470.1 | 1 | 33/63 |
| T13-T14 | K.TVEGAGSIAAATGFVK.K | 1479.66001 | 0.97 | 6.01 | 1342.6 | 1 | 23/45 |
| Human CSF | | | | | | | Peptide |
| Trypsin-generated | Peptide fragment sequence | | | Score | MW | Accession | (Hits) |
| peptide fragment # | Column eluate #3 | MH+ | Sf | XC | Sp | RSp | Ions |
| | Alpha-synuclein isoform | | 4.96 | 60.32 | 14460.1 | 4507109 | (230000) |
| | NACP140 | | | | | | |
| T3-T4 | K.AKEGVVAAAEK.T | 1072.59970 | 0.65 | 2.61 | 305.3 | 1 | 16/30 |
| T7-T8 | K.TKEGVLYVGSK.T | 1181.36357 | 0.85 | 2.91 | 839.2 | 1 | 18/30 |
| T9-T10 | K.EGVVHGVATVAEK.T | 1295.69539 | 0.75 | 2.92 | 258.8 | 1 | 19/36 |
| T10 | K.EGVVHGVATVAEK.T | 1296.45466 | 0.88 | 3.28 | 962.9 | 1 | 22/36 |
| T11-T12 | K.TKEVTNVGGAVVTGVTAVAQK.T | 2158.44170 | 0.97 | 4.74 | 1549.2 | 1 | 32/63 |
| T12 | K.EQVTNVGGAVVTGVTAVAQK.T | 1929.16446 | 0.75 | 3.11 | 407.0 | 1 | 20/57 |
| T13-T14 | K.TGVGASSIAAATGFVK.K | 1479.86001 | 0.97 | 5.15 | 1857.8 | 1 | 25/45 |

Amino acids are listed in their letter codes; bold letters indicate human sequence-specific residues.
Note, eluates #2 and #3 also contained 146 additional peptides corresponding to a total of 58 distinct
proteins, which were present in LD-CSF and had been enriched together with human alpha-synuclein.
These included known CSF constituents (e.g., cystatin C), hypothetical proteins of unknown function,
and several neural proteins that await further validation as true interactors of alpha-synuclein (e.g.,
prion protein, tau-tubulin kinase) (for details, see Mollenhauer and Schlossmacher, 2008).

Development of second-generation ELISAs for alpha-synuclein quantification

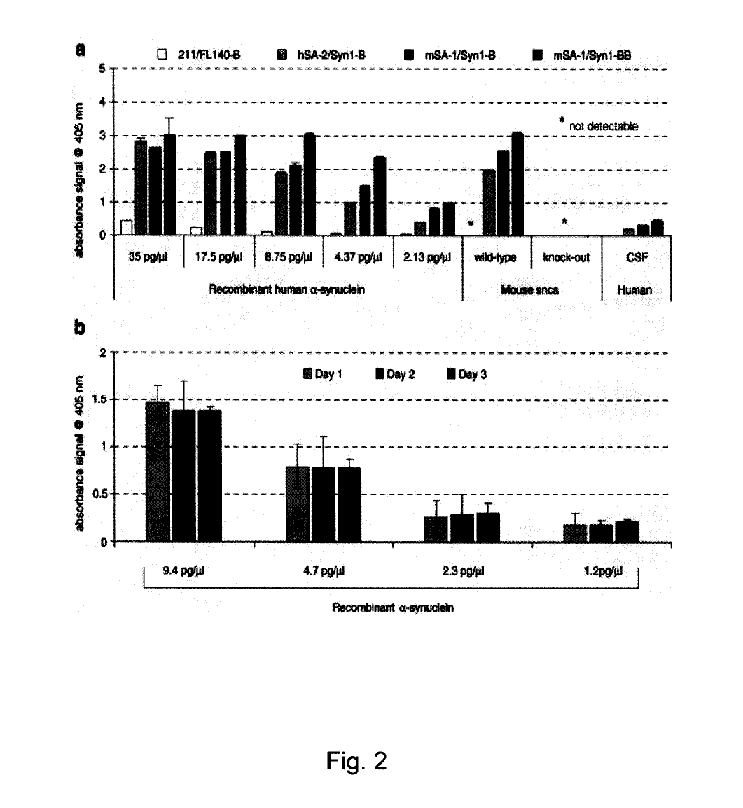

We previously had to concentrate CSF to reliably detect alpha-synuclein signals (Tokuda et al., 2006), because our first-generation ELISA [211/FL140-B] did not convincingly react with native, (i.e., unconcentrated) CSF, as shown in FIG. 2a.

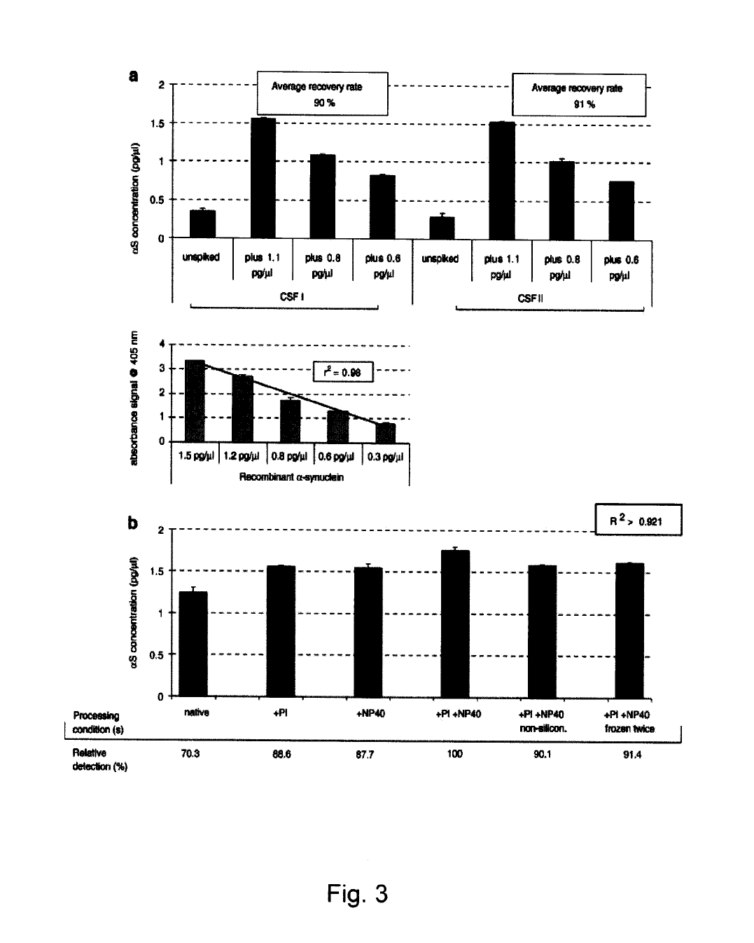

Following the generation of three alternate antibody (Ab) pairs, i.e., [7071AP/Syn1-B], [hSA-2/Syn1-B] and [mSA-1/Syn1-B], we further validated each sandwich by using wild-type versus snca knock-out mouse specimens (Abeliovich et al., 2000) (FIG. 2a). In related immunodepletion experiments, in antibody titration studies and in serial ELISA repeats, we next addressed the issues of sensitivity, specificity and signal variability. There, we found that the new ELISAs were highly specific for alpha-synuclein and demonstrated day-to-day (plate-to-plate) signal variability of <10% (FIG. 2b). There, we consistently recorded data (in now >70 ELISA plates analyzed) that featured high signal reproducibility and linear performance in the low pg range (FIG. 3).

### Optimization of CSF Processing and ELISA Protocols

Following the confirmation of signal specificity and reproducibility, we optimized the sensitivity of these ELISAs in detecting alpha-synuclein from CSF and introduced final modifications to the assaying arm (Materials and methods). We focused on the ELISA sandwich [mSA-1/Syn1-B], which consistently generated stronger OD signals than all other pairs. As a result of these biochemical modifications, the CSF alpha-synuclein-signal-to-background ratio was now enhanced >10-fold over the first-generation ELISA [211/FL140-B] (Tokuda et al., 2006), as demonstrated for example in FIG. 2a (compare white with black bars for three sources of alpha-synuclein). The sensitivity of the novel ELISA, which we refer to as [mSA-1/Syn1-BB] from hereon, ranged as low as 0.1 ng/ml (or, pg/μl) of human alpha-synuclein.

As demonstrated in FIG. 3a, this ELISA consistently featured a >90% recovery rate of human alpha-synuclein from CSF, as demonstrated in serial spiking experiments with recombinant protein. In practice, this optimized ELISA protocol now permitted for the first time the direct measurement of CSF alpha-synuclein. Hence, these collective modifications reduced the number of processing steps, lessened the chance for protein loss or error, and minimized signal variability.

We next determined the ideal conditions for CSF processing in preparation for ELISA. As shown in FIG. 3b, we found that the addition of protease inhibitors and NP40 detergent consistently increased OD signal strength by up to 30%. We also found that the use of non-siliconized tubes and two freeze/thaw cycles reduced the ELISA signal by ˜10% each (FIG. 3b).

### Cross-Sectional Examination of CSF Alpha-Synuclein by ELISA

For a first pilot study that directly examined unconcentrated CSF by this new ELISA, we selected specimens from twenty age-matched, previously untested living donors from four diagnostic groups, i.e., PD, AD, DLB, and control subjects. Using a 96-well format for the ELISA, we recorded lower mean CSF alpha-synuclein levels in the PD subjects (n=5) and DLB donors (n=6) when compared with age- and gender-matched, neurologically intact control subjects (n=4) and AD patients (n=5; data not shown). A Monte Carlo approximated permutation test for the variable, CSF alpha-synuclein, between the two cohorts of greatest interest in this study, PD and DLB (representing parkinsonism with suspected synucleinopathy) versus AD and controls (subjects without parkinsonism), was significant (p=0.0305), despite the small size of the cohort.

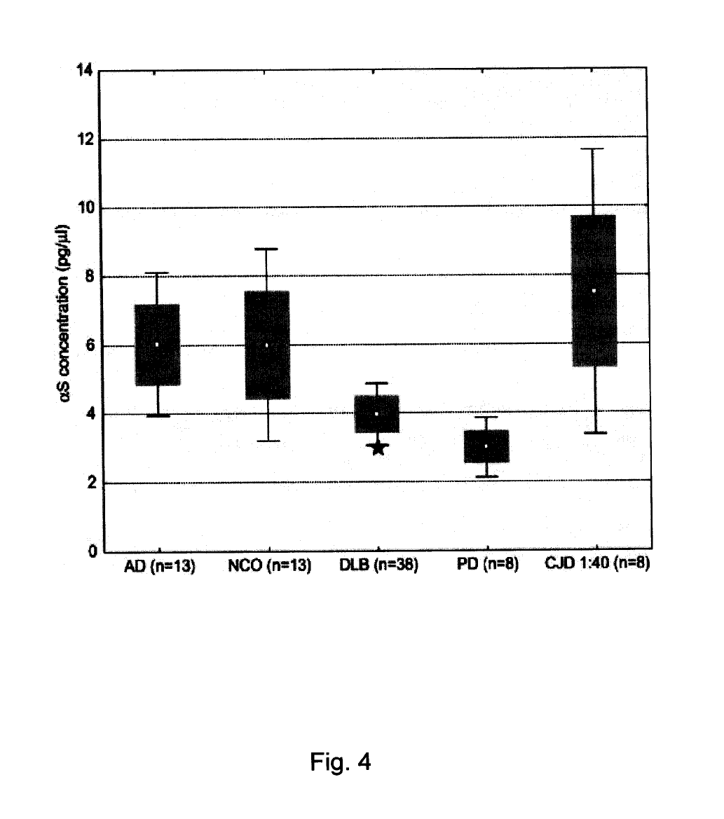

We next carried out a larger, cross-sectional study using our 384-well ELISA protocol. We examined unconcentrated CSF specimens from a randomly chosen cohort of 72 new subjects of Caucasian background that encompassed the following categories: AD, DLB, PD and a group that we classified as ‘other neurological controls’ (NCO) representing non-neurodegenerative diagnoses. We also chose a fifth group, i.e., eight patients with definite CJD, where we anticipated a higher concentration of intra-neuronal proteins to appear in CSF. As shown in FIG. 4, we recorded a sizeable overlap in individual signals between the AD, NCO, PD and DLB donors, but nevertheless registered lower mean CSF alpha-synuclein concentrations in the synucleinopathy cases when compared with the non-synucleinopathy donors (FIG. 4). In contrast, CSF alpha-synuclein concentrations were markedly elevated in all CJD subjects, as expected. The latter CSF specimens were also positive for the 14-3-3 proteins (not shown). The group values for these five diagnostic categories (n=80), their age distribution, duration of illness prior to lumbar puncture, gender ratio and CSF protein concentrations are summarized in Table 2. There, we also calculated the ratio for the ‘CSF alpha-synuclein concentration-to-CSF total protein content’ because the pool of proteins in human CSF is subject to change depending on age, brain volume and hydrodynamics (Bateman et al., 2006 and Reiber, 2003), and because we observed that the range of the CSF total protein concentration varied between the five disease categories (Table 2).

TABLE 2
Summary of demographics and CSF alpha-synuclein values of donors shown in FIG. 4.

| | Alzheimer | Neurological | Dementia with | Parkinson | Creutzfeldt- |
|---|---|---|---|---|---|
| | disease | controls | Lewy bodies | disease | Jakob disease |
| | (AD) | (NCO) | (DLB) | (PD) | (CJD) |
| Number of patients | 13 | 13 | 38 | 8 | 8 |
| Sex [male/total] | 0.3 | 0.6 | 0.6 | 0.7 | 0.4 |
| Age [years] | | | | | |
| Mean ± SD | 69 ± 9 | 64 ± 15 | 71 ± 8 | 76 ± 3 | 71 ± 7 |
| (Range) | (57-80) | (32-79) | (59-84) | (73-83) | (59-79) |
| Median | 70 | 68 | 73 | 75 | 72 |
| Duration of disease | | | | | |
| [months] | | | | | |
| Mean ± SD | 51 ± 36 | n.d. | 26 ± 29 | 100 ± 58 | 5 ± 3 |
| (Range) | (36-122) | | (14-120) | (48-200) | (4-12) |
| Median | 42 | | 18 | 84 | 4 |
| CSF total protein | | | | | |
| conctration [mg/ml] | | | | | |
| Mean ± SD | 0.5 ± 0.3 | 0.9 ± 0.8 | 0.7 ± 0.3 | 0.6 ± 0.2 | 0.3 ± 0.1 |
| (Range) | (0.2-1.2) | (0.4-3.0) | (0.1-1.2) | (0.4-0.9) | (0.2-0.4) |
| Median | 0.5 | 0.7 | 0.7 | 0.7 | 0.3 |
| CSF alpha-synuclein | | | | | (*) |
| [pg/μl] | | | | | |
| Mean ± SD | 6.2 ± 4.2 | 6.0 ± 5.7 | 3.8 ± 3.3 | 3.0 ± 1.3 | 300 ± 248 |
| (Range) | (1.9-16.1) | (2.0-16.2) | (0.8-16.0) | (1.3-5.4) | (77-607) |
| Median | 4.2 | 3.0 | 3.0 | 3.11 | 164.1 |
| Ratio, CSF alpha- | | | | | (*) |
| synuclein-to-total protein | | | | | |
| concentration [pg/mg] | | | | | |
| Mean ± SD | 17.4 ± 16.2 | 7.0 ± 4.4 | 6.0 ± 4.6 | 4.9 ± 3.1 | 1000 ± 935 |
| (Range) | (3.0-26.9) | (2.8-18.5) | (0.9-18.0) | (2.3-11.9) | (176-2761) |
| Median | 11.0 | 5.9 | 4.5 | 4.3 | 492.0 |

SD = standard deviation;
(*) = after correction of 1:40 dilution.
Note, the group of neurological controls (non-neurodegenerative diseases) included subjects with: small vessel disease-related strokes (n = 4); chronic inflammatory demyelinating polyradiculopathy (n = 3); paraneoplastic syndromes (n = 2); acute headaches (n = 1); normal pressure hydrocephalus (n = 1); pseudotumor cerebri (n = 1); and cervical myelopathy (n = 1).

### Statistical Analyses of CSF Alpha-Synuclein in Prion Disease and Synucleinopathy Groups

To analyze the degree of separation in CSF alpha-synuclein concentrations between CJD cases and other groups, we performed a Satterthwaite approximate T-test. There, the difference between the individual CJD versus NCO concentrations was statistically significant, even after adjustment for the lower CSF protein content (Table 2) in all CJD cases (p<0.001). From the perspective of the clinically diagnosed synucleinopathies, we determined that the CSF alpha-synuclein level and the afore-mentioned variable, ‘CSF alpha-synuclein concentration-to-CSF total protein concentration’, were significantly different in the combined PD and DLB groups when compared with the NCO and AD groups (p=0.025; p=0.0018, respectively). However, in the same four categories we noted an inverse association between donor age and CSF alpha-synuclein concentration (Table 2) and an age difference between the two combined cohorts (p=0.0341). Therefore, to adjust for co-varying age and also for any difference in the CSF protein content among these groups we carried out ANCOVA testing, which confirmed that the mean ‘log transformed CSF alpha-synuclein concentration’ was significantly lower in the DLB and PD cases than in the NCO and AD subjects (p<0.001). Logarithmic transformation was performed to meet assumptions of normality and homogenous group variances.

### Autopsy Validation of Clinical Diagnoses

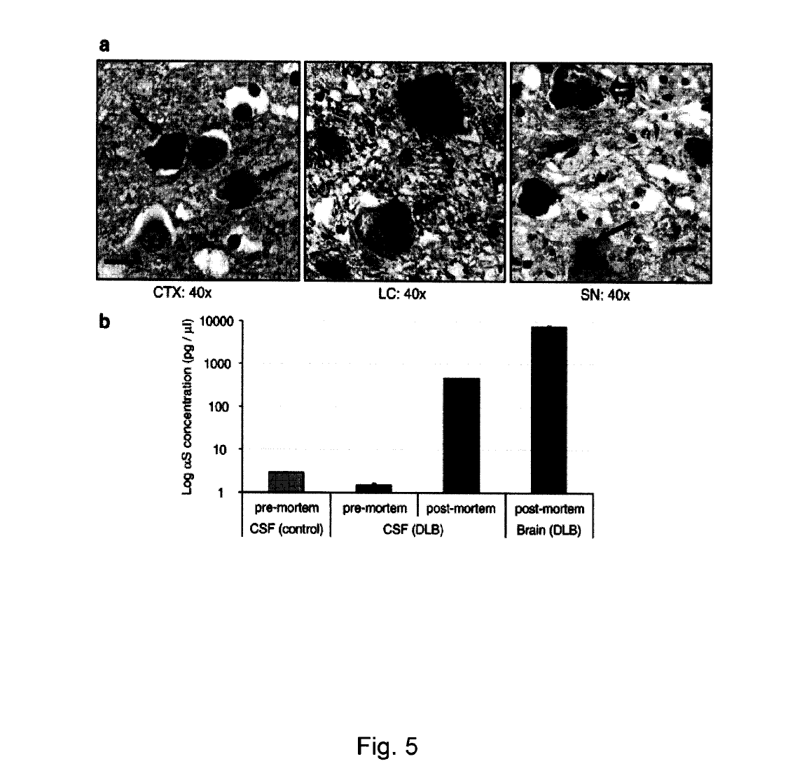

To date, we have learned of nine deaths among the 100 CSF donors that we analyzed for this study. As predicted from their clinical diagnosis and their positive 14-3-3 tests, all patients with suspected CJD have died. At autopsy, the clinical diagnosis of prion disease was confirmed in each case (data not shown). In one patient from the DLB group, the working diagnosis of a primary synucleinopathy was confirmed by immunohistochemistry at autopsy, which revealed all the hallmarks of brainstem and cortical Lewy body disease (Jellinger, 2003) (FIG. 5a). These autopsy findings therefore validated the accuracy of the clinical diagnoses in 9% of all donors enrolled in this study and in 11.25% of the 80 donors shown in FIG. 4.

### Rapid Cell Death Promotes Rise in CSF Alpha-Synuclein

ELISA quantification of three available specimens from our index patient with definite DLB, i.e., intra vitam CSF, post mortem CSF and a specimen from frontal cortex, revealed alpha-synuclein concentrations of 2.9, 876.0 and 9430 pg/μl, respectively (FIG. 5b). We next validated the marked elevation in CSF alpha-synuclein following cell death in an independent cohort of autopsy-confirmed PD, AD, Huntington disease and neurologically healthy control cases (n=12). There, the average ventricular concentration of CSF alpha-synuclein measured 1275±1044 pg/μl at a mean ventricular CSF collection time of 15.2 h after death. In these twelve cases, CSF alpha-synuclein concentrations steadily increased with the length of the post mortem interval (i.e., autolysis). In contrast, the CSF alpha-synuclein did not correlate well with the degree of hemolysis. Without wishing to be bound by theory, our combined results from living CJD cases and post mortem studies suggested to us that a pathological rise in CSF alpha-synuclein is seen during rapid neurodegeneration and cell lysis, most likely because of the release of heretofore intracellular constituents (e.g., 14-3-3, tau) (Mollenhauer et al., 2003).

### Discussion

Using complementary protocols to detect and measure human alpha-synuclein, we present several findings: one, we demonstrate by affinity enrichment and MS that alpha-synuclein is indeed a constituent of cell-free CSF obtained from a neurologically intact, adult donor. Previously, its presence had been inferred (El-Agnaf et al., 2003, Tokuda et al., 2006 and van Geel et al., 2008), but not yet demonstrated by sequencing analysis. We calculated that the detectable CSF alpha-synuclein concentration in vivo is equal to <0.001% of the total CSF proteome. Of note, in related efforts we also discovered that β- and γ-synucleins are constituents of adult human CSF (Mollenhauer et al., 2006b). Although we did not yet identify the two most C-terminal fragments of alpha-synuclein in our tryptic digests (Table 1), our Western blotting results (El-Agnaf et al., 2003) and the strong reactivity of our affinity-purified antibodies against the C-terminus (Periquet et al., 2007) suggest that at least a portion of CSF alpha-synuclein contains the full-length protein (FIG. 1). Of note, our newly raised polyclonal antibodies (7071AP; hSA-2; mSA-1) and by inference, our second-generation ELISA pairs, are specific for alpha-synuclein proteins from rodent and primate sources (FIG. 2a) but—to our knowledge—not for any particular post-translational modification; they react with both monomeric and soluble oligomeric alpha-synuclein species (Periquet et al., 2007) (I.K., J.N., M.G.S., unpublished data) but do not detect β- or γ-synuclein, as shown for example, by the lack of ELISA signal in alpha-synuclein-deficient mouse tissue (FIG. 2a). Further exploration of adult human CSF by MS and the use of modification-specific antibodies are now required to characterize and quantify all detectable variants of alpha-synuclein in human CSF. Such an approach was recently employed by Anderson et al studying uniquely modified alpha-synuclein species in synucleinopathy-rich human brain (Anderson et al., 2006).

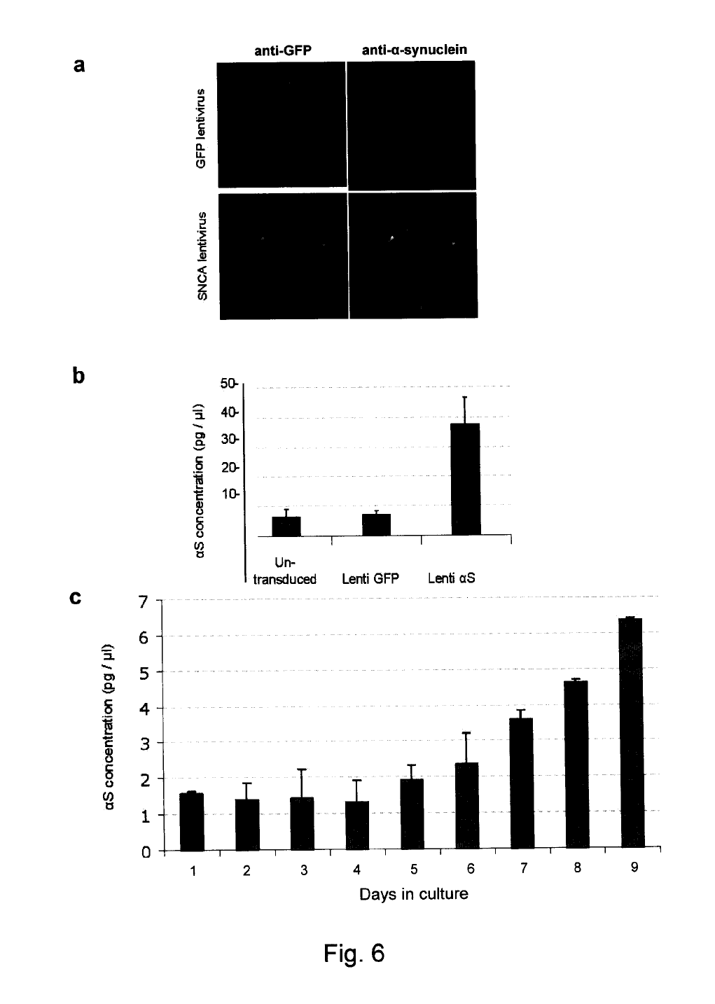

Two, in related work, we found that alpha-synuclein can be detected in cell-, debris- and serum-free conditioned media of primary neuronal cultures in the absence of any apparent injury (FIG. 6). Previously, investigators have postulated a release by intact neuroblastoma and select non-neural cells, but neurons had not yet been analyzed. To address this, we introduced human SNCA cDNA by lentivirus (SNCA-LV) into cortical cells from rat and mouse embryos (FIG. 6a). The serum-free conditioned medium (CM) of mature neurons was analyzed by ELISA and contained human alpha-synuclein. The latter correlated well with the amount of SNCA cDNA expressed and occurred without apparent cell death, as examined by three established toxicity assays (not shown). Moreover, we recorded the specific appearance of endogenous rat alpha-synuclein in the CM of mature cortical neurons without viral transduction (FIG. 6c). These data suggested to us that minute amounts of alpha-synuclein are present in neural fluids ex vivo. However, the concept of a ‘physiological secretion’ of alpha-synuclein remains controversial given the absence of an endoplasmic reticulum leader sequence for the nascent protein (El-Agnaf et al., 2003 and Lee et al., 2005) and because of its close association with vesicles near the plasma membrane (Barbour et al., 2008 and Li et al., 2002). An alternative explanation for the presence of extracellular alpha-synuclein in apparently intact cells is lysosomal exocytosis or membrane blebbing. Of note, Keller et al recently described an additional mechanism, which sees the secretion of leaderless proteins by intact cells in a caspase 1-dependent manner (Keller et al., 2008). In the future, our optimized ELISA [hSA-2/Syn1-B] protocol for cellular alpha-synuclein may help delineate the responsible mechanism(s) underlying the appearance of alpha-synuclein in extracellular fluids.

In vivo, there are four principal contributors to the CSF proteome: one, the secretion of proteins during neural metabolism (e.g., Aβ, APP) (Seubert et al., 1992); two, their release by the epithelium of the choroid plexus (e.g., cystatin C) (Chodobski and Szmydynger-Chodobska, 2001); three, a low level entry rate of soluble blood constituents following their filtration (e.g., serum albumin) (Chodobski and Szmydynger-Chodobska, 2001 and Reiber, 2003); and four, the release of cellular constituents during ageing-related natural attrition (e.g., tau) (Fagan et al., 2007, Mollenhauer et al., 2006a, Mollenhauer et al., 2005b and Motter et al., 1995). Intriguingly, in related immunohistochemistry work, we detected specific, diffuse staining of the choroid plexus epithelium within human ventricles by two well characterized, monoclonal anti-alpha-synuclein antibodies (J.P.V.S., I.P., M.G.S., unpublished data). Our collective results suggest that the level of alpha-synuclein in adult human CSF is regulated by several sources, and that the relative contribution from each varies depending on the clinical situation. Specifically, we envision at least three possible scenarios: In the first, the level of CSF alpha-synuclein steadily declines during ageing under physiological conditions (Tokuda et al., 2006). In the second, pathologically high CSF alpha-synuclein levels occur during rapid neurodegeneration, as demonstrated in our CJD cases and post mortem analyses (FIGS. 4 and 5). In the third scenario, CSF alpha-synuclein levels may be raised because of a compromise in the blood brain barrier. Several groups, including ours, have provided evidence for the presence of alpha-synuclein in human blood products, i.e., erythrocytes, reticulocytes, platelets, serum and plasma (Barbour et al., 2008, El-Agnaf et al., 2003, El-Agnaf et al., 2006, Fjorback et al., 2007 and Miller et al., 2004). By examining venous blood from healthy adult donors by ELISA, we recently calculated the following mean alpha-synuclein concentrations: in hemolysis-free serum, the mean was 25.0±0.9 pg/μl; in cell-free plasma, 45.0±0.1 pg/μl; and in whole blood lysates, 24.1±1.7 ng/μl (Scherzer C R et al. 2008; Proc Nat Acad Sci; in press). Therefore, the naturally higher concentration of alpha-synuclein in plasma could theoretically affect the corresponding CSF alpha-synuclein level in the case of either systemic or nervous system-wide inflammation (see below).

Three, we present the development, optimization and validation of a second-generation ELISA protocol that permits for the first time the direct quantification of alpha-synuclein in native CSF, and concurrently, in other extracellular fluids (Schlossmacher, 2006 and van Geel et al., 2008). We validated the specificity of CSF alpha-synuclein detection by ELISA in several ways: by anti-alpha-synuclein depletion studies (Mollenhauer and Schlossmacher, 2008); by a >90% recovery rate for alpha-synuclein in serial spiking experiments using recombinant, human alpha-synuclein (FIG. 3a); and by the use of snca-null mouse brain and blood products (FIG. 2a). The improvement over our previously reported first-generation ELISA can be explained by enhanced sensitivity of the affinity-purified, antibodies (mSA-1; hSA-2) and the specificity of the monoclonal antibody (Syn-1) used for antigen detection (FIG. 2a). Here, we calculated that the concentration of CSF alpha-synuclein in our neurological control specimens ranged from 2.0-16.2 pg/μl (Table 2). We had previously estimated that in concentrated specimens the mean CSF alpha-synuclein level was slightly higher, i.e., >20 pg/μl (Tokuda et al., 2006). Our explanation for this apparent discrepancy is that two different sources for the standard reference protein, i.e., recombinant, human full-length alpha-synuclein, were used. Here, we also identified conditions that lead to reduced CSF alpha-synuclein signals during processing at 4° C. (FIG. 3b). Clearly, for future comparisons of CSF alpha-synuclein concentrations among different cohorts, it will be imperative to adopt and then record the implementation of rigorously standardized operating procedures across participating centers, and to follow strict quality control guidelines for the use of recombinant standards and antibodies (Fagan et al., 2007 and Rimm, 2006).

Four, we present a first cross-sectional quantification of alpha-synuclein in unconcentrated CSF from patients with a wide range of neurological conditions. Among our 100 donors, 83% suffered from a neurodegenerative illness; 57% were clinically diagnosed with a primary synucleinopathy; 18% had a primary tau-related disorder; and 8% had definite prion disease. Despite their small number, the diagnosis of CJD was unequivocally established by 14-3-3 testing intra vitam and later by autopsy, thereby meeting the highest validation standard possible (Cummings et al., 2008 and Grossman et al., 2005). We found a very marked elevation in CSF alpha-synuclein in living CJD subjects over all other diagnostic groups (FIG. 4; Table 2), which was statistically highly significant. Prospective studies will determine whether CSF alpha-synuclein is of any value in the earlier diagnosis of prion disease than is currently accomplished through 14-3-3 testing in CSF (Otto et al., 2002 and Wadsworth et al., 2006).

Five, in the same feasibility study our data reveal a marginal but significant difference in the mean concentration of CSF alpha-synuclein in patients with parkinsonism linked to clinically diagnosed DLB and PD versus control groups (FIG. 4; Table 2). Our non-CJD cohort was randomly chosen from a CSF bank that was built by employing widely used diagnostic criteria. The difference between the primary synucleinopathy group versus control cohorts remained statistically significant even after correcting for age variability, after removing CJD patients from our analyses, and after correction of the CSF protein content. With respect to the latter, in our non-neurodegenerative NCO group, the CSF protein content was highest among donors diagnosed with a paraneoplastic syndrome, followed by those with acute headache and chronic inflammatory demyelinating polyradiculopathy (all ≧0.98 mg/ml), when compared with those suffering, for example, from microangiopathic strokes. The corresponding CSF alpha-synuclein concentration ranked highest in subjects with acute headache, paraneoplastic syndrome and normal pressure hydrocephalus (all ≧6.7 pg/μl; Table 2). These findings suggested to us that in this relatively small cohort of 13 NCO donors, which encompassed only four subjects with inflammatory conditions (Table 2), a possible compromise of the blood brain barrier during inflammation did not consistently elevate the CSF alpha-synuclein concentration.

Overall, we recorded a substantial overlap in individual CSF alpha-synuclein concentrations among donors within all groups excluding CJD cases (Table 2). Such signal overlap has been observed in other cross-sectional investigations of CSF markers, for example, in the quantification of CSF amyloid β protein and tau isoforms (Blennow and Hampel, 2003, Fagan et al., 2007 and Motter et al., 1995). Therefore, we performed a logistic regression analysis of this cross-sectional cohort, which indicated that the ratio of ‘CSF alpha-synuclein-to-total protein’ in combination with age provided significant prediction of the diagnosis of synucleinopathy versus non-synucleinopathy (p<0.001); there, the area under the ROC curve (sensitivity plotted against 1 minus specificity) equaled 0.72, and each one provided significant prediction beyond that afforded by the other (p<0.05). With this report we have now recorded a relatively low mean CSF alpha-synuclein concentration in donors with presumed synucleinopathy in a second, independent study. The former featured exclusively PD patients, the current one has a substantially higher number of DLB than PD subjects (Table 2). Our teams employed two different protocols, i.e., ELISA [mSA-1/Syn1-BB] here versus [21]/FL140-B] there, examined a Caucasian and an Asian cohort, analyzed unconcentrated and concentrated CSF, and performed these studies in two separate laboratories (Boston, USA; Al Ain, UAE), respectively (Tokuda et al., 2006). The next validation phase will include larger cross-sectional cohorts with additional analyses regarding the specificity and sensitivity of CSF alpha-synuclein in predicting the diagnosis of PD, DLB (and possibly, MSA). Longitudinal, prospective studies are also planned to quantify CSF alpha-synuclein in early versus advanced stages of parkinsonism, combined with the assessment of other CSF values and the detailed recording of clinical parameters for motor and cognitive performances in each donor (Bibi et al., 2006, Mollenhauer et al., 2005a, Mollenhauer et al., 2006a and Mollenhauer et al., 2006c).

The question for a mechanism underlying the apparent ‘reduction’ of total CSF alpha-synuclein in our synucleinopathy cases remains intriguing, but nevertheless premature. A satisfactory answer will require the establishment of the normal concentration range in CSF from neurologically and medically healthy donors processed under the same conditions reported here. Our current investigational protocol did not yet permit the collection of CSF from an entirely healthy, adult control population. Another limitation of our CSF alpha-synuclein-focused exploration is the potentially confounding effect of pharmacotherapy, for example by L-dopa itself (Fahn et al., 2004). Mignini et al. (2000) have described the expression of dopamine receptors in the choroid plexus (Mignini et al., 2000), which upon activation could alter CSF alpha-synuclein homeostasis in medicated patients with parkinsonism. It is therefore imperative to examine whether CSF alpha-synuclein concentrations differ in drug-naive versus treated patients, and to resolve whether mammalian models of progressive intracellular synucleinopathy (reviewed in Chesselet, 2008) feature reduced CSF alpha-synuclein levels. Lastly, measuring CSF alpha-synuclein in clinically affected and unaffected carriers of SNCA gene mutations (Fuchs et al., 2007, Fuchs et al., 2008 and Nishioka et al., 2006) should provide further insights into the mechanisms that underlie our observations.

### 2. Cerebrospinal Fluid Values in Patients with Synucleinopathies

Intracellular accumulation of α-synuclein is a hallmark feature of synucleinopathy disorders of the brain that include Parkinson disease (PD), dementia with Lewy bodies (DLB), and multiple system atrophy (MSA). In Experiment 1 above the quantification of total α-synuclein in cerebrospinal fluid (CSF alpha-synuclein) by enzyme-linked immunoabsorbent assay (ELISA) in patients with neurodegenerative disorders is discussed. Using a first and then second generation ELISA system in concentrated and native CSF, respectively, we recorded a reduction in mean CSF alpha-synuclein values from PD patients when compared with Alzheimer disease (AD) patients and other neurological controls.

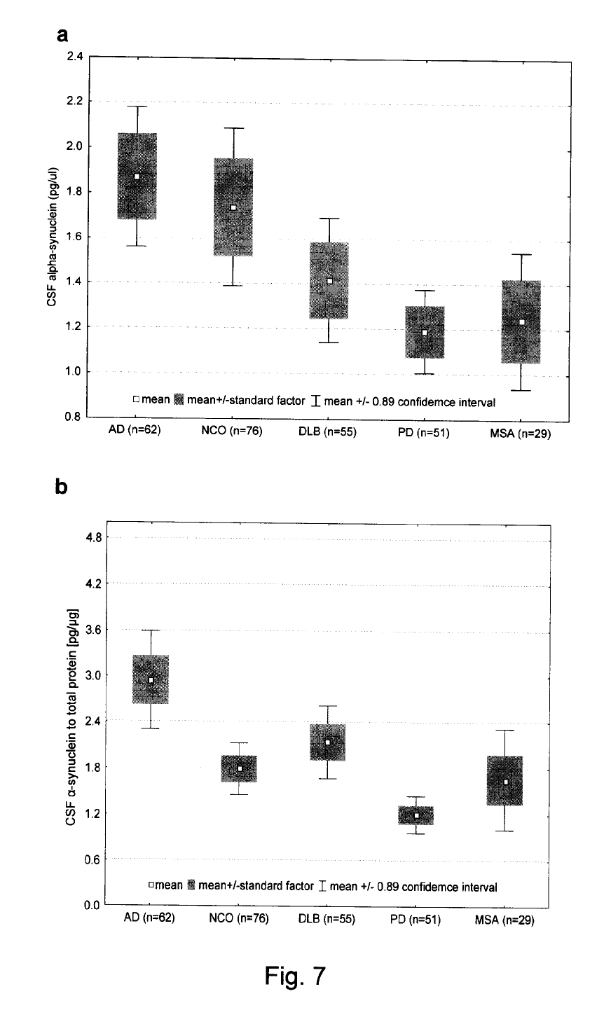

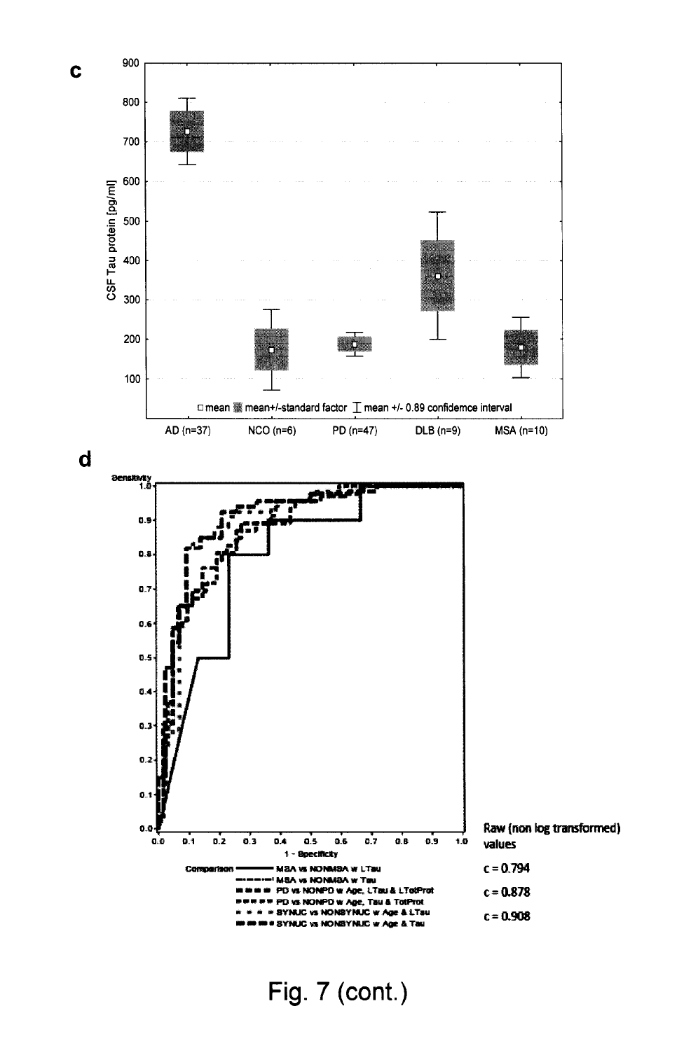

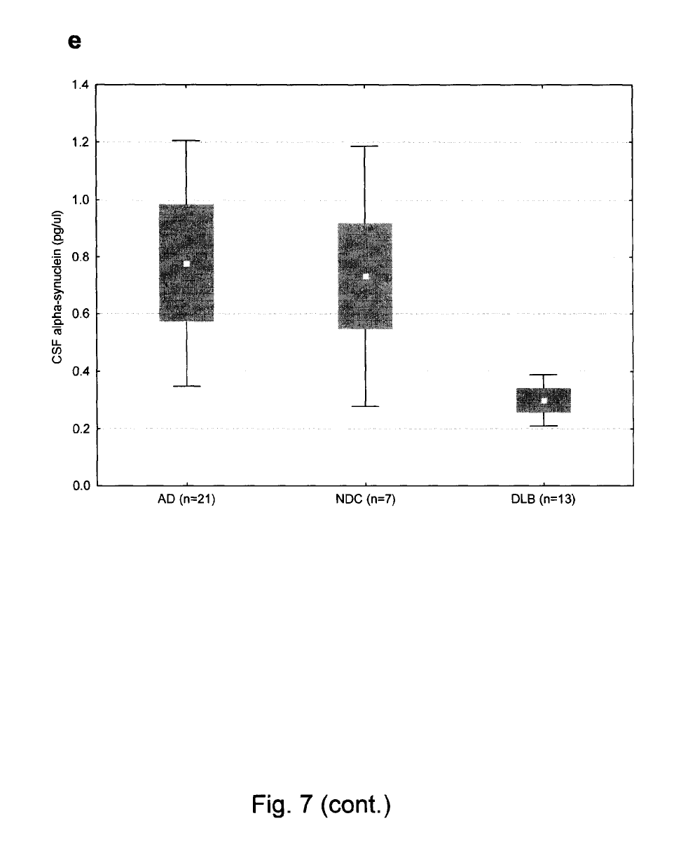

To subject CSF alpha-synuclein quantification to validation as a possible marker for intracellular synucleinopathies other than PD (such as MSA and DLB), we conducted a large cross-sectional study comprising 273 Caucasians from five distinct diagnostic groups (Table 3; FIG. 7). CSF specimens were collected, processed and analyzed for values of CSF alpha-synuclein, CSF tau, and for the total CSF protein concentration.

In these 273 subjects (62 AD, 76 NCO, 55 DLB, 51 PD, 29 MSA), the logarithmically transformed values for the absolute CSF alpha-synuclein concentration and its relative value (i.e., CSF alpha-synuclein divided by CSF protein level) showed significant differences between the diagnostic groups (p=0.0156 and p=0.0006, respectively) in ANCOVA studies. Because we detected that the CSF protein concentration differed (p=0.0002) between the diagnostic groups (Table 3) after adjustment for age, we also carried out an ANCOVA with log CSF alpha-synuclein as the dependent variable and where age and log CSF total protein were simultaneous covariates. A significant omnibus diagnostic effect (p=0.0006) was followed up with Tukey post-hoc tests which indicated that adjusted mean CSF alpha-synuclein values were significantly (or marginally) different between PD versus AD (p=0.0002), PD versus NCO (p=0.0921), MSA versus AD (p=0.0793), and DLB versus AD(p=0.0912). In logistic regression analyses, we found that a combination of CSF alpha-synuclein and CSF total protein values, when adjusted for age, significantly discriminated PD donors from subjects with AD and NCO (omnibus p<0.0001) at a combined sensitivity and specificity [area under the receiver operating curve (AUC)) value of c=0.849. However, subjects with DLB and MSA were at best only marginally discriminated from the AD/NCO donor group (c=0.699 and 0.654, respectively). The combination of all donors with suspected synucleinopathy into a single group (PD/DLB/MSA) were discriminated even less well (AUC=0.712) than the combination of PD and MSA (AUC=0.779), from the non-synucleinopathy group of AD/NCO donors, most likely because of the intermediate value for DLB (FIG. 7a-b).

However, adding log(and non-log transformed) CSF tau concentration levels to the log(and non-log transformed) CSF alpha-synuclein discriminated the diagnostic groups best and significantly, primarily discriminating ADs from DLB (p=0.003), MSA, NCO and PD (p<0.001) and to a lesser degree DLB from these latter groups (MSA: p=0.0023; NCO: p=0.464 and PD: p=0.127). The combination of all donors with suspected synucleinopathy into a single group (PD/DLB/MSA) was discriminated well (AUC=0.908) from the non-synucleinopathy group of AD/NCO donors, most likely because of the elevation of total tau in AD donors (FIG. 7c-d).

To address any pharmacological effects of L-dopa therapy (Table 3) on the steady-states of either CSF alpha-synuclein concentration or CSF protein levels (or both), we next examined a subgroup of our PD patients without any levodopa therapy (n=2), without levodopa 12 hours before lumbar puncture (n=3) and without time released levodopa over night (n=13), but did not find any significance (p=0.39 for CSF alpha-synuclein and p=0.75 for CSF total protein). Due to outliers and small and disparate numbers in each group we ran a Wilcoxon Rank Sum Test and a distribution free raw score permutation test. The same was done for DLB patients with any levodopa medication within 30 days prior to lumbar puncture (n=10) and those without any levodopa at all (n=24) (p=0.0696 for CSF alpha-synuclein and p=0.0588 for CSF protein).

We also carried out validation studies in yet another independent cohort of 41 CSF donors with neurodegeneration to pursue autopsy confirmation of probable DLB donors; this, because DLB subjects represent a common source of diagnostic inaccuracy in living subjects with cognitive impairment due to the frequent co-occurrence of AD pathology and because of the controversy in the field over the value of CSF alpha-synuclein in probable DLB subjects. Our results revealed the biologically expected overlap in individual CSF values between different categories of subjects with cognitive impairment, but nevertheless indicated lower mean CSF alpha-synuclein concentrations in our group of autopsy-confirmed DLB cases (n=13) when compared with definite AD and NDC donors (n=28; FIG. 7e; Table 4).

TABLE 4

| | Alzheimer | Neurological | Dementia with |
|---|---|---|---|
| | Disease | Disease Controls | Lewy Bodies |
| | (AD) | (NDC) | (DLB) |
| Number of patients | 21 | 7 | 13 |
| Sex [male/total] | 0.5 | 0.7 | 0.5 |
| Age [years] | | | |
| Mean ± SD | 67 ± 16 | 61 ± 6 | 72 ± 12 |
| (range) | (35-93) | (49-69) | (36-87) |
| CSF α-synuclein | | | |
| concentration [pg/μl] | | | |
| Mean ± SD | 0.8 ± 0.9 | 0.7 ± 0.5 | 0.3 ± 0.2 |
| (range) | (0.2-3.8) | (0.3-1.5) | (0.1-0.7) |

To determine the degree of separation using biochemical CSF values in this series of 41 subjects with definite causes of dementia, we carried out ANCOVA analysis using the logarithmically transformed value of CSF alpha-synuclein. There, the difference between the DLB and NDC groups revealed significance (p=0.0148). The comparison between DLB and AD was also significant as analyzed by a Satterthwaite approximate t-test (p=0.0326). Logistic regression of the probability of carrying the diagnosis of DLB versus AD as a function of CSF alpha-synuclein was significant with an AUC value of c=0.69 (p=0.0190). In contrast, logistic regression with CSF tau—either alone or together with CSF alpha-synuclein—revealed no significance (p=˜0.21 and p=0.23, respectively).

In summary, we analyzed second and third cross-sectional cohorts of patients with the clinical diagnosis of a progressive neurodegenerative disorder. We again recorded a significant reduction in the absolute and relative values for CSF alpha-synuclein in patients with PD and in subjects with MSA. Of note, four of these 29 MSA patients were diagnosed with the cerebellar variant (MSA-C), 25 with the parkinsonian form of MSA (MSA-P). In this small sample, no difference was detected in CSF alpha-synuclein between MSA-C and MSA-P donors.

Importantly, DLB patients showed a significant decrease of CSF alpha-synuclein in our independent clinical and autopsy confirmed groups. Nevertheless DLB remains heterogeneous, which could be due to the overlap with AD both clinically and neuropathologically. It is unclear to date why DLB patients with more advanced alpha-synuclein pathology have higher CSF levels compared to our other synucleinopathies (PD and MSA), but we speculate that it may be linked to more widespread degeneration of synapses and neurons.

TABLE 3
Demographic data for clinical subjects undergoing lumbar puncture.

| | | | Dementia | | Multiple | |
|---|---|---|---|---|---|---|
| | Alzheimer | Neurological | with Lewy | Parkinson | system | |
| | disease | controls | Bodies | disease | atrophy | |
| | (AD) | (NCO)¹ | (DLB) | (PD) | (MSA) | p |
| | probable diagnosis | | | | | value |
| Number of patients | 62 | 76 | 55 | 54 | 29 | |
| Sex [male/total] | 0.4 | 0.4 | 0.5 | 0.6 | 0.6 | n.s. |
| Age [years] | | | | | | |
| Mean ± SD | 69 ± 10 | 54 ± 21 | 71 ± 7 | 73 ± 7 | 67 ± 7 | |
| (range) | (55-86) | (25-90) | (55-85) | (49-83) | (52-77) | |
| Duration of disease | | | | | | |
| [months] | | | | | | |
| Mean ± SD | 30 ± 18 | na | 27 ± 17 | 146 ± 77.20 | 35 ± 20 | |
| (range) | (12-72) | | (13-90) | (24-312) | (12-84) | |
| Equivalent dosage of | | | | | | |
| levodopa² [mg] | | | | | | |
| Mean ± SD | 0 | 0 | 107 ± 220 | 723 ± 424 | 295 ± 370 | |
| (range) | | | (0-1,065) | (0-1,800) | (0-1,215) | |
| CSF tau protein [pg/ml] | | | | | | |
| (number of donors) | 37 | 6 | 9 | 47 | 10 | |
| Mean ± SD | 772 ± 365 | 174 ± 129 | 539 ± 338 | 189 ± 125 | 179 ± 151 | |
| (range) | (178-1265) | (75-373) | (75-850) | (75-626) | (75-450) | |
| CSF total protein | | | | | | |
| concentration [μg/μl] | | | | | | |
| Mean ± SD | 0.7 ± 0.2 | 0.9 ± 0.5 | 0.7 ± 0.3 | 1.0 ± 0.4 | 0.8 ± 0.1 | 0.0002 |
| (range) | (0.2-1.2) | (0.4-3.1) | (0.1-1.8) | (0.6-3.2) | (0.5-1.1) | |
| CSF α-synuclein | | | | | | |
| concentration [pg/μl] | | | | | | |
| Mean ± SD | 1.9 ± 1.5 | 1.7 ± 1.8 | 1.4 ± 1.3 | 1.2 ± 0.8 | 1.2 ± 1.0 | 0.0156 |
| (range) | (0.5 -7.3) | (0.5-7.4) | (0.5-7.2) | (0.5-4.7) | (0.7-6.2) | |
| Ratio, CSF α-synuclein- | | | | | | |
| to- total protein concentration | | | | | | |
| [pg/μg] | | | | | | |
| Mean ± SD | 2.8 ± 2.5 | 1.8 ± 1.5 | 2.1 ± 1.8 | 1.2 ± 0.9 | 1.7 ± 1.7 | |
| (range) | (0.6-12.1) | (0.5-8.2) | (0.3-9.3) | (0.4-6.3) | (0.8-10.4) | |

¹The group of neurological controls included subjects with headache (n = 32), peripheral neuropathy and chronic inflammatory demyelinating polyradiculitis (n = 9), depression (n = 6), focal cerebrovascular disease (n = 5), epilepsy (n = 4), pseudotumor cerebri (n = 6), amyotrophic lateral sclerosis (n = 3), acute myelitis (n = 1), multiple sclerosis (n = 2), paraneoplastic syndrome (n = 1), normal pressure hydrocephalus (n = 2), brain tumor (n = 2), systemic lupus (n = 1), meningeal carcinomatosis (n = 1).
²Equivalency dosages were calculated according to established guidelines (Oertel et al., 2003)

### 3. Concentration of α-Synuclein in Biological Fluids, including EDTA-Drawn Whole Blood, Plasma, Serum CSF Compared to Engineered Cell Lysates

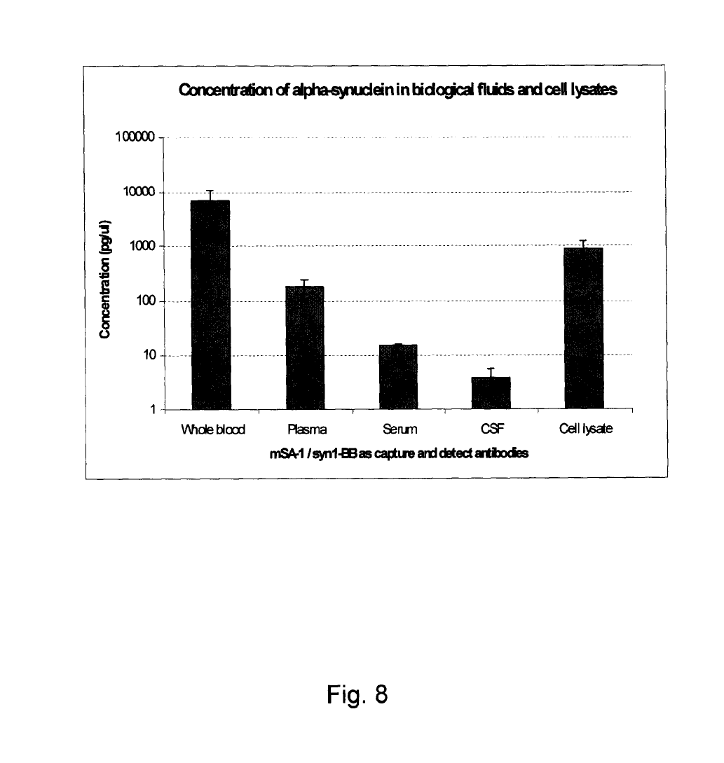

Levels of α-synuclein were measured in several biological fluids including EDTA-drawn whole blood, plasma, serum, and CSF from the same donor controlling for the total protein concentration of all specimens (shown in comparison with lysates of neuronal cells that over-express human, wild-type alpha-synuclein) using the above-described methods. The results of testing are shown in FIG. 8, in which it is clear that the CSF alpha-synuclein concentration is lowest in CSF as compared to EDTA whole blood, plasma, and serum. In the group of normal adult donors, alpha-synculein concentrations measured >15.0 pg/ul in fresh serum, >50.0 pg/ul in fresh plasma, and ˜10 ng/ul in whole blood lysates.

### 4. Quantification of α-Synuclein in Venous Blood from a Newly Established, Longitudinal Case-Control Cohort

We set out to conduct a longitudinal biomarker study for Parkinson disease (PD) at two large, Medical School-affiliated hospitals in Boston, Mass. and to quantify alpha synuclein (aSyn) in venous blood from three age-matched donor groups. We hypothesized that aSyn values are dysregulated in subjects with sporadic PD when compared with age-matched healthy (HCO) and neurological controls (NCO).

ASyn is an abundantly expressed, neuronal and hematological protein that is linked to the development of PD by genetics and neuropathology. Increased levels of SNCA mRNA and aSyn protein in the brain and venous blood have been identified in subjects with rare, heritable forms of PD [Miller D W et al., Neurology 2004]. Recent feasibility and cross-sectional studies demonstrated that aSyn concentrations in venous blood [El-Agnaf et al., FASEB J 2006; Barbour R et al., Neurodegen Dis 2008; Scherzer C et al., PNAS 2008] and cerebrospinal fluid [Tokuda T et al., BBRC 2006; Mollenhauer B et al., Exp Neurol 2008] represent candidate values in the exploration of biological markers for PD and related neurodegenerative disorders. Here, we sought to determine the total concentration of aSyn in venous blood from well characterized subjects at the time of enrollment into a longitudinal study.

To this end, we established a longitudinal, case-control cohort of PD subjects diagnosed according to the UKPDS Brain Bank criteria (Hughes J et al., 1995), of age-matched HCO subjects and of NCO donors diagnosed with neurodegenerative conditions other than PD. We performed neurological examinations on PD and NCO subjects, recorded UPDRS, HY and MMSE scores, and obtained EDTA complete blood counts and reticulocyte numbers from each donor. We collected serum, plasma and whole EDTA blood specimens by routine phlebotomy for aSyn measurements that were determined by a recently well characterized sandwich ELISA method [Mollenhauer et al., Exp Neurol 2008; Scherzer C et al., PNAS 2008; Cullen V et al. Mol Brain 2009]. Blood products were drawn by vacutainer, processed within eight hours of phlebotomy, and stored at minus 80° C. as serum, plasma (following centrifugation at 5,000 rpm for 10 min) or whole EDTA blood lysates prior to ELISA analysis. We and others determined recently that >99 percent of aSyn protein in peripheral blood can be found within erythrocytes, reticulocytes and platelets when compared with the <1 percent that is contained within the extracellular milieu (FIG. 8) [Barbour R et al., 2008; Scherzer C R et al., 2008].

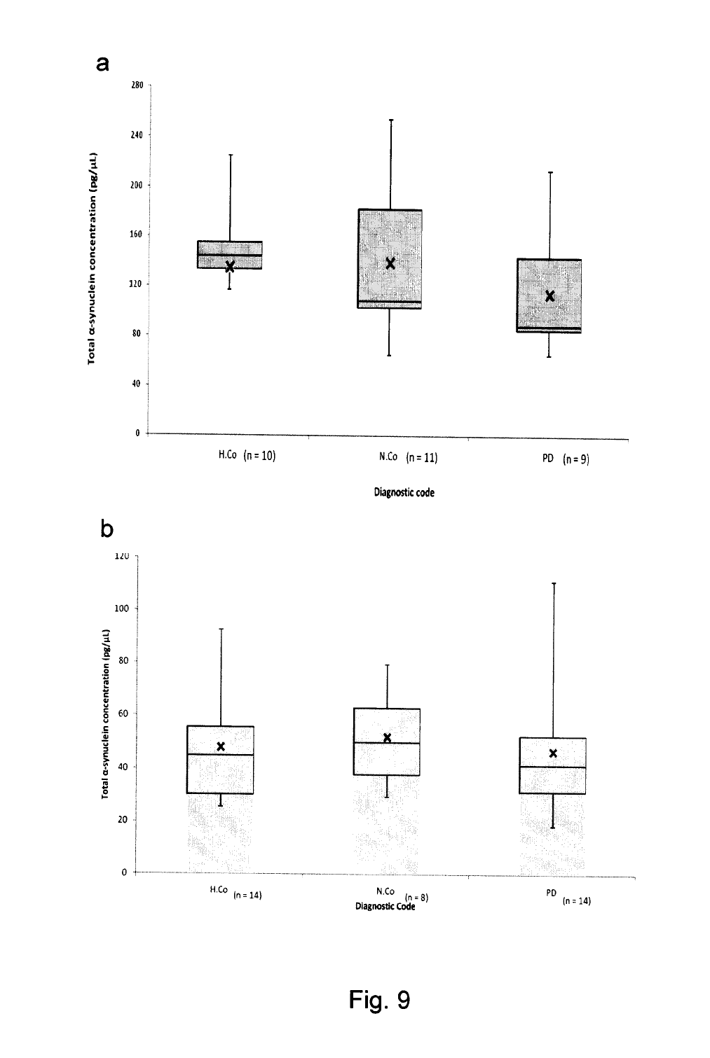

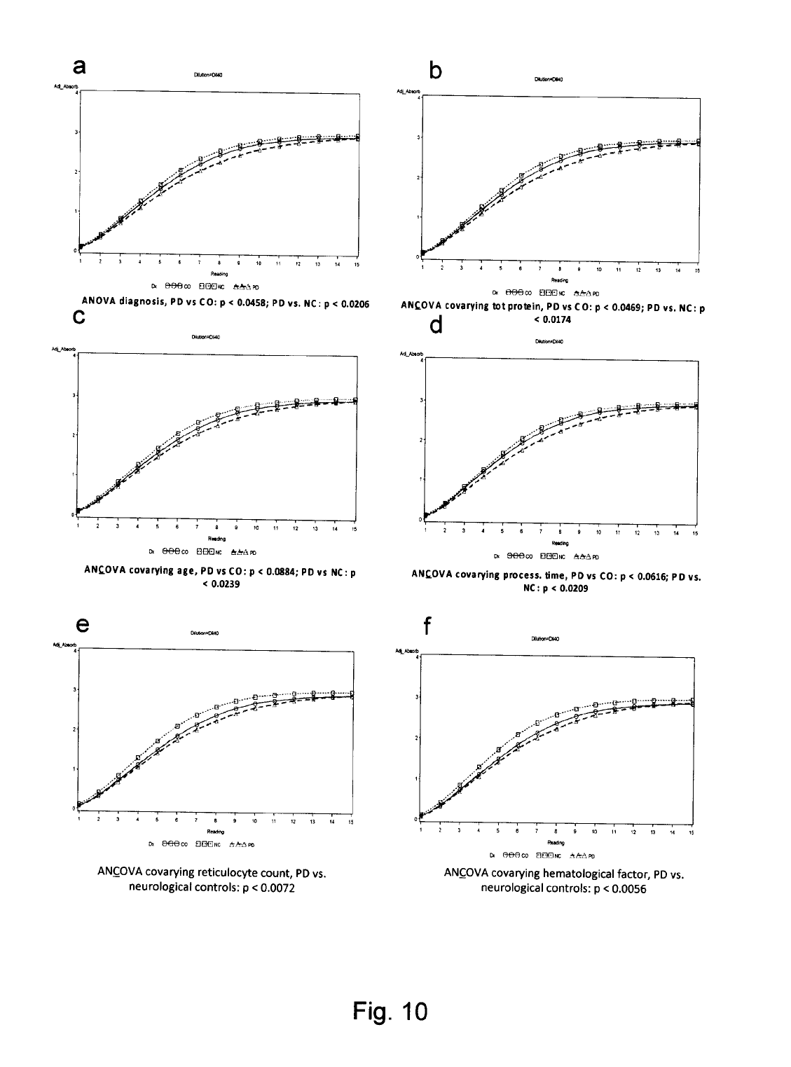

We enrolled 319 donors comprising age-matched PD (n=160; 50%), NCO (n=74; 23%) and HCO subjects (n=85; 27%) (FIG. 9). Our results revealed a statistically significant reduction of total plasma alpha-synuclein in PD donors when compared with HCO (p<0.04) and NCO (p<0.02) subjects by ANOVA. ANCOVA testing to covary the total protein content of plasma confirmed the significant reduction of alpha-synuclein in PD vs NCO patients (p<0.017; PD vs HCO, p<0.046). ANCOVA testing to covary age and the processing time after phlebotomy confirmed the significant reduction of alpha-synuclein in plasma from PD donors (FIG. 8c-d). Finally, ANCOVA testing to covary the reticulocyte count (FIG. 8e) and the aggregate hematological factor (FIG. 8f) best distinguished the reduction of plasma alpha-synuclein in PD vs NCO patients (p<0.0072; p<0.0065).

Representative examples of ELISA plates monitoring plasma α-synuclein levels in patients with Parkinson's (PD) versus healthy controls and neurological controls from 319 donors at the time of enrollment, and from 80 donors at the time of 1-year follow up are shown in FIG. 9a-b. Whisker Plots (FIG. 9) show the mean plasma alpha-synuclein levels are reduced in both sample sets.

### 5. Protocol to Collect Proteome, RNA, DNA and ztube for CBC(PROBE)

The following protocol was used for patient sampling during clinical studies:

Samples 1, 2, and 3 (PAXgene): Store upright at room temp for 24 hours, then transfer to a −20 C Freezer prior to shipment on dry ice.

Sample 4 (4 mL SST): Centrifuge SST and transfer 1 mL of serum to yellow cap eppendorf tube. Write “S” on the yellow cap. Freeze serum in a −80 Freezer until ready to ship to the University of Ottawa on dry ice.

Sample 5 (4 mL EDTA): Ship to Reference Lab at ambient temperature on day of collection.

Sample 6 (4 mL EDTA): Freeze whole blood from 4 mL EDTA in a −80 Freezer until ready to ship to the University of Ottawa on dry ice.

Sample 7 (10 mL EDTA): Centrifuge 10 mL EDTA tube centrifuge at 5,000 rpm at 4 degrees Celsius (10 minutes). Transfer 1 mL plasma to each of 4 purple cap eppendorf tubes. Write “L” on the purple cap of the last eppendorf tube filled. Freeze cells remaining in 10 mL tube and four plasma aliquots in a −80 Freezer until ready to ship to the University of Ottawa on dry ice.

### REFERENCES

Abeliovich et al., 2000 A. Abeliovich, Y. Schmitz, I. Farinas, D. Choi-Lundberg, W. H. Ho, P. E. Castillo, N. Shinsky, J. M. G. Verdugo, M. Armanini and A. Ryan, Mice lacking [alpha]-synuclein display functional deficits in the nigrostriatal dopamine system, Neuron 25 (2000), pp. 239-252.
Anderson et al., 2006 J. P. Anderson, D. E. Walker, J. M. Goldstein, R. de Laat, K. Banducci, R. J. Caccavello, R. Barbour, J. Huang, K. Kling, M. Lee, L. Diep, P. S. Keim, X. Shen, T. Chataway, M. G. Schlossmacher, P. Seubert, D. Schenk, S. Sinha, W. P. Gai and T. J. Chilcote, Phosphorylation of Ser 129 is the dominant pathological modification of alpha-synuclein in familial and sporadic Lewy body disease, J. Biol. Chem. 281 (2006), pp. 29739-29752.
Andreasen et al., 1999 N. Andreasen, C. Hesse, P. Davidsson, L. Minthon, A. Wallin, B. Winblad, H. Vanderstichele, E. Vanmechelen and K. Blennow, Cerebrospinal fluid beta-amyloid(1-42) in Alzheimer disease: differences between early- and late-onset Alzheimer disease and stability during the course of disease, Arch. Neurol. 56 (1999), pp. 673-680.
Barbour et al., 2008 R. Barbour, K. Kling, J. P. Anderson, K. Banducci, T. Cole, L. Diep, M. Fox, J. M. Goldstein, F. Soriano, P. Seubert and T. J. Chilcote, Red blood cells are the major source of alpha-synuclein in blood, Neurodegener Dis. 5 (2008), pp. 55-59.
Bateman et al., 2006 R. J. Bateman, L. Y. Munsell, J. C. Morris, R. Swarm, K. E. Yarasheski and D. M. Holtzman, Human amyloid-beta synthesis and clearance rates as measured in cerebrospinal fluid in vivo, Nat. Med. 12 (2006), pp. 856-861.
Bibl et al., 2006 M. Bibl, B. Mollenhauer, H. Esselmann, P. Lewczuk, H. W. Klafki, K. Sparbier, A. Smirnov, L. Cepek, C. Trenkwalder, E. Ruther, J. Komhuber, M. Otto and J. Wiltfang, CSF amyloid-beta-peptides in Alzheimer's disease, dementia with Lewy bodies and Parkinson's disease dementia, Brain 129 (2006), pp. 1177-1187.
Blennow and Hampel, 2003 K. Blennow and H. Hampel, CSF markers for incipient Alzheimer's disease, Lancet Neurol. 2 (2003), pp. 605-613.
Chesselet, 2008 M. F. Chesselet, In vivo alpha-synuclein overexpression in rodents: a useful model of Parkinson's disease?, Exp. Neurol. 209 (2008), pp. 22-27.
Chodobski and Szmydynger-Chodobska, 2001 A. Chodobski and J. Szmydynger-Chodobska, Choroid plexus: target for polypeptides and site of their synthesis, Microsc. Res. Tech. 52 (2001), pp. 65-82.
Conway et al., 2000 K. A. Conway, S. J. Lee, J. C. Rochet, T. T. Ding, R. E. Williamson and P. T. Lansbury Jr., Acceleration of oligomerization, not fibrillization, is a shared property of both alpha-synuclein mutations linked to early-onset Parkinson's disease: implications for pathogenesis and therapy, Proc. Natl. Acad. Sci. U.S.A. 97 (2000), pp. 571-576.
Cookson, 2005 M. R. Cookson, The biochemistry of Parkinson's disease, Annu. Rev. Biochem. 74 (2005), pp. 29-52.
Cookson and van der Brug, 2008 M. R. Cookson and M. van der Brug, Cell systems and the toxic mechanism(s) of alpha-synuclein, Exp. Neurol. 209 (2008), pp. 5-11.
Cuervo et al., 2004 A. M. Cuervo, L. Stefanis, R. Fredenburg, P. T. Lansbury and D. Sulzer, Impaired degradation of mutant alpha-synuclein by chaperone-mediated autophagy, Science 305 (2004), pp. 1292-1295.
Cummings et al., 2008 J. Cummings, T. H. Ward, A. Greystoke, M. Ranson and C. Dive, Biomarker method validation in anticancer drug development, Br. J. Pharmacol. 153 (2008), pp. 646-656.
Dorsey et al., 2006 E. R. Dorsey, R. G. Holloway and B. M. Ravina, Biomarkers in Parkinson's disease, Expert. Rev. Neurother. 6 (2006), pp. 823-831.
Dubois et al., 2007 B. Dubois, H. H. Feldman, C. Jacova, S. T. Dekosky, P. Barberger-Gateau, J. Cummings, A. Delacourte, D. Galasko, S. Gauthier, G. Jicha, K. Meguro, J. O'Brien, F. Pasquier, P. Robert, M. Rossor, S. Salloway, Y. Stem, P. J. Visser and P. Scheltens, Research criteria for the diagnosis of Alzheimer's disease: revising the NINCDS-ADRDA criteria, Lancet Neurol. 6 (2007), pp. 734-746.
El-Agnaf et al., 1998 O. M. El-Agnaf, R. Jakes, M. D. Curran, D. Middleton, R. Ingenito, E. Bianchi, A. Pessi, D. Neill and A. Wallace, Aggregates from mutant and wild-type alpha-synuclein proteins and NAC peptide induce apoptotic cell death in human neuroblastoma cells by formation of beta-sheet and amyloid-like filaments, FEBS Lett. 440 (1998), pp. 71-75.
El-Agnaf et al., 2003 O. M. El-Agnaf, S. A. Salem, K. E. Paleologou, L. J. Cooper, N. J. Fullwood, M. J. Gibson, M. D. Curran, J. A. Court, D. M. Mann, S. Ikeda, M. R. Cookson, J. Hardy and D. Allsop, Alpha-synuclein implicated in Parkinson's disease is present in extracellular biological fluids, including human plasma, Faseb J. 17 (2003), pp. 1945-1947.
El-Agnaf et al., 2006 O. M. El-Agnaf, S. A. Salem, K. E. Paleologou, M. D. Curran, M. J. Gibson, J. A. Court, M. G. Schlossmacher and D. Allsop, Detection of oligomeric forms of alpha-synuclein protein in human plasma as a potential biomarker for Parkinson's disease, Faseb J. 20 (2006), pp. 419-425.
Fagan et al., 2007 A. M. Fagan, C. M. Roe, C. Xiong, M. A. Mintun, J. C. Morris and D. M. Holtzman, Cerebrospinal fluid tau/beta-amyloid(42) ratio as a prediction of cognitive decline in nondemented older adults, Arch. Neurol. 64 (2007), pp. 343-349.
Fahn et al., 2004 S. Fahn, D. Oakes, I. Shoulson, K. Kieburtz, A. Rudolph, A. Lang, C. W. Olanow, C. Tanner and K. Marek, Levodopa and the progression of Parkinson's disease, N. Engl. J. Med. 351 (2004), pp. 2498-2508.
Farrer, 2006 M. J. Farrer, Genetics of Parkinson disease: paradigm shifts and future prospects, Nat. Rev. Genet. 7 (2006), pp. 306-318.
Fjorback et al., 2007 A. W. Fjorback, K. Vanning and P. H. Jensen, Determination of alpha-synuclein concentration in human plasma using ELISA, Scand J. Clin. Lab. Invest. 67 (2007), pp. 431-435.
Fuchs et al., 2007 J. Fuchs, C. Nilsson, J. Kachergus, M. Munz, E. M. Larsson, B. Schule, J. W. Langston, F. A. Middleton, O. A. Ross, M. Hulihan, T. Gasser and M. J. Farrer, Phenotypic variation in a large Swedish pedigree due to SNCA duplication and triplication, Neurology 68 (2007), pp. 916-922.
Fuchs et al., 2008 J. Fuchs, A. Tichopad, Y. Golub, M. Munz, K. J. Schweitzer, B. Wolf, D. Berg, J. C. Mueller and T. Gasser, Genetic variability in the SNCA gene influences {alpha}-synuclein levels in the blood and brain, Faseb J 22 (5) (2008), pp. 1327-1334.
Gai et al., 1998 W. P. Gai, J. H. Power, P. C. Blumbergs and W. W. Blessing, Multiple-system atrophy: a new alpha-synuclein disease?, Lancet 352 (1998), pp. 547-548.
Gibb, 1988 W. R. Gibb, Accuracy in the clinical diagnosis of parkinsonian syndromes, Postgrad. Med. J 64 (1988), pp. 345-351.
Grossman et al., 2005 M. Grossman, J. Farmer, S. Leight, M. Work, P. Moore, V. Van Deerlin, D. Pratico, C. M. Clark, H. B. Coslett, A. Chatterjee, J. Gee, J. Q. Trojanowski and V. M. Lee, Cerebrospinal fluid profile in frontotemporal dementia and Alzheimer's disease, Ann. Neurol. 57 (2005), pp. 721-729.
Halliday and McCann, 2008 G. M. Halliday and H. McCann, Human-based studies on alpha-synuclein deposition and relationship to Parkinson's disease symptoms, Exp. Neurol. 209 (2008), pp. 12-21.
Jellinger, 2003 K. A. Jellinger, Neuropathological spectrum of synucleinopathies, Mov. Disord. 18 (Suppl 6) (2003), pp. S2-12.
Keller et al., 2008 M. Keller, A. Ruegg, S. Werner and H. D. Beer, Active caspase-1 is a regulator of unconventional protein secretion, Cell 132 (2008), pp. 818-831.
Klein and Schlossmacher, 2006 C. Klein and M. G. Schlossmacher, The genetics of Parkinson disease: implications for neurological care, Nat. Clin. Pract. Neurol. 2 (2006), pp. 136-146.
Kramer and Schulz-Schaeffer, 2007 M. L. Kramer and W. J. Schulz-Schaeffer, Presynaptic alpha-synuclein aggregates, not Lewy bodies, cause neurodegeneration in dementia with Lewy bodies, J. Neurosci. 27 (2007), pp. 1405-1410.
Lee et al., 2005 H. J. Lee, S. Patel and S. J. Lee, Intravesicular localization and exocytosis of alpha-synuclein and its aggregates, J. Neurosci. 25 (2005), pp. 6016-6024.
Lewczuk et al., 2006 P. Lewczuk, G. Beck, H. Esselmann, R. Bruckmoser, R. Zimmermann, M. Fiszer, M. Bibl, J. M. Maier, J. Kornhuber and J. Wiltfang, Effect of sample collection tubes on cerebrospinal fluid concentrations of tau proteins and amyloid beta peptides, Clin. Chem. 52 (2006), pp. 332-334.
Li et al., 2002 Q. X. Li, B. C. Campbell, C. A. McLean, D. Thyagarajan, W. P. Gai, R. M. Kapsa, K. Beyreuther, C. L. Masters and J. G. Culvenor, Platelet alpha- and gamma-synucleins in Parkinson's disease and normal control subjects, J. Alzheimers Dis. 4 (2002), pp. 309-315.
Li et al., 2007 Q. X. Li, S. S. Mok, K. M. Laughton, C. A. McLean, R. Cappai, C. L. Masters, J. G. Culvenor and M. K. Horne, Plasma alpha-synuclein is decreased in subjects with Parkinson's disease, Exp. Neurol. 204 (2007), pp. 583-588.
McKeith et al., 1996 I. G. McKeith, D. Galasko, K. Kosaka, E. K. Perry, D. W. Dickson, L. A. Hansen, D. P. Salmon, J. Lowe, S. S. Mirra, E. J. Byrne, G. Lennox, N. P. Quinn, J. A. Edwardson, P. G. Ince, C. Bergeron, A. Burns, B. L. Miller, S. Lovestone, D. Collerton, E. N. Jansen, C. Ballard, R. A. de Vos, G. K. Wilcock, K. A. Jellinger and R. H. Perry, Consensus guidelines for the clinical and pathologic diagnosis of dementia with Lewy bodies (DLB): report of the consortium on DLB international workshop, Neurology 47 (1996), pp. 1113-1124.
McKeith et al., 2005 I. G. McKeith, D. W. Dickson, J. Lowe, M. Emre, J. T. O'Brien, H. Feldman, J. Cummings, J. E. Duda, C. Lippa, E. K. Perry, D. Aarsland, H. Arai, C. G. Ballard, B. Boeve, D. J. Burn, D. Costa, T. Del Ser, B. Dubois, D. Galasko, S. Gauthier, C. G. Goetz, E. Gomez-Tortosa, G. Halliday, L. A. Hansen, J. Hardy, T. Iwatsubo, R. N. Kalaria, D. Kaufer, R. A. Kenny, A. Korczyn, K. Kosaka, V. M. Y. Lee, A. Lees, I. Litvan, E. Londos, O. L. Lopez, S. Minoshima, Y. Mizuno, J. A. Molina, E. B. Mukaetova-Ladinska, F. Pasquier, R. H. Perry, J. B. Schulz, J. Q. Trojanowski, M. Yamada and for the Consortium on DLB, Diagnosis and management of dementia with Lewy bodies: Third report of the DLB consortium, Neurology 65 (2005), pp. 1863-1872.
McKhann et al., 1984 G. McKhann, D. Drachman, M. Folstein, R. Katzman, D. Price and E. M. Stadlan, Clinical diagnosis of Alzheimer's disease: report of the NINCDS-ADRDA Work Group under the auspices of Department of Health and Human Services Task Force on Alzheimer's Disease, Neurology 34 (1984), pp. 939-944.
Michell et al., 2004 A. W. Michell, S. J. Lewis, T. Foltynie and R. A. Barker, Biomarkers and Parkinson's disease, Brain 127 (2004), pp. 1693-1705.
Mignini et al., 2000 F. Mignini, E. Bronzetti, L. Felici, A. Ricci, M. Sabbatini, S. K. Tayebati and F. Amenta, Dopamine receptor immunohistochemistry in the rat choroid plexus, J. Auton Pharmacol. 20 (2000), pp. 325-332.
Miller et al., 2004 D. W. Miller, S. M. Hague, J. Clarimon, M. Baptista, K. Gwinn-Hardy, M. R. Cookson and A. B. Singleton, Alpha-synuclein in blood and brain from familial Parkinson disease with SNCA locus triplication, Neurology 62 (2004), pp. 1835-1838.
Mollenhauer et al., 2003 B. Mollenhauer, S. Serafin, I. Zerr, B. J. Steinhoff, M. Otto, M. Scherer, W. J. Schulz-Schaeffer and S. Poser, Diagnostic problems during late course in Creutzfeldt-Jakob disease, J. Neurol. 250 (2003), pp. 629-630.
Mollenhauer et al., 2005a B. Mollenhauer, M. Bibl, C. Trenkwalder, G. Stiens, L. Cepek, P. Steinacker, B. Ciesielczyk, K. Neubert, J. Wiltfang, H. A. Kretzschmar, S. Poser and M. Otto, Follow-up investigations in cerebrospinal fluid of patients with dementia with Lewy bodies and Alzheimer's disease, J. Neural. Transm. 112 (2005), pp. 933-948.
Mollenhauer et al., 2005b B. Mollenhauer, L. Cepek, M. Bibl, J. Wiltfang, W. J. Schulz-Schaeffer, B. Ciesielczyk, M. Neumann, P. Steinacker, H. A. Kretzschmar, S. Poser, C. Trenkwalder and M. Otto, Tau protein, Abeta42 and S-100B protein in cerebrospinal fluid of patients with dementia with Lewy bodies, Dement. Geriatr. Cogn. Disord. 19 (2005), pp. 164-170.
Mollenhauer et al., 2006a B. Mollenhauer, M. Bibl, J. Wiltfang, P. Steinacker, B. Ciesielczyk, K. Neubert, C. Trenkwalder and M. Otto, Total tau protein, phosphorylated tau (181p) protein, beta-amyloid(1-42), and beta-amyloid(1-40) in cerebrospinal fluid of patients with dementia with Lewy bodies, Clin. Chem. Lab. Med. 44 (2006), pp. 192-195.
Mollenhauer et al., 2006b B. Mollenhauer, B. Krastins, C. Trenkwalder, M. G. Schlossmacher and D. A. Sarracino, Proteome analysis of cerebrospinal fluid by mass spectrometry: a platform for marker development in synucleinopathies, Movement Disorders 21 (Suppl 15) (2006).
Mollenhauer et al., 2006c B. Mollenhauer, C. Trenkwalder, N. von Ahsen, M. Bibl, P. Steinacker, P. Brechlin, J. Schindehuette, S. Poser, J. Wiltfang and M. Otto, Beta-amyloid 1-42 and tau-protein in cerebrospinal fluid of patients with Parkinson's disease dementia, Dement. Geriatr. Cogn. Disord. 22 (2006), pp. 200-208.
Mollenhauer and Schlossmacher, 2008 B. Mollenhauer and M. Schlossmacher, Purification and quantification of neural alpha-synuclein: Relevance for pathogenesis and biomarker development. In: R. Nass and S. Przedborski, Editors, Parkinson's Disease: Pathogenic and Therapeutic Insights, vol. 41, Elsevier Academic Press Inc. (2008), pp. 559-574.
Motter et al., 1995 R. Motter, C. Vigo-Pelfrey, D. Kholodenko, R. Barbour, K. Johnson-Wood, D. Galasko, L. Chang, B. Miller, C. Clark and R. Green et al., Reduction of beta-amyloid peptide42 in the cerebrospinal fluid of patients with Alzheimer's disease, Ann. Neurol. 38 (1995), pp. 643-648.
Nishioka et al., 2006 K. Nishioka, S. Hayashi, M. J. Farrer, A. B. Singleton, H. Yoshino, H. Imai, T. Kitami, K. Sato, R. Kuroda, H. Tomiyama, K. Mizoguchi, M. Murata, T. Toda, I. Imoto, J. Inazawa, Y. Mizuno and N. Hattori, Clinical heterogeneity of alpha-synuclein gene duplication in Parkinson's disease, Ann. Neurol. 59 (2006), pp. 298-309.
Otto et al., 2002 M. Otto, J. Wiltfang, L. Cepek, M. Neumann, B. Mollenhauer, P. Steinacker, B. Ciesielczyk, W. Schulz-Schaeffer, H. A. Kretzschmar and S. Poser, Tau protein and 14-3-3 protein in the differential diagnosis of Creutzfeldt-Jakob disease, Neurology 58 (2002), pp. 192-197.
Outeiro and Lindquist, 2003 T. F. Outeiro and S. Lindquist, Yeast cells provide insight into alpha-synuclein biology and pathobiology, Science 302 (2003), pp. 1772-1775.
Periquet et al., 2007 M. Periquet, T. Fulga, L. Myllykangas, M. G. Schlossmacher and M. B. Feany, Aggregated alpha-synuclein mediates dopaminergic neurotoxicity in vivo, J. Neurosci. 27 (2007), pp. 3338-3346.
Polymeropoulos et al., 1997 M. H. Polymeropoulos, C. Lavedan, E. Leroy, S. E. Ide, A. Dehejia, A. Dutra, B. Pike, H. Root, J. Rubenstein, R. Boyer, E. S. Stenroos, S. Chandrasekharappa, A. Athanassiadou, T. Papapetropoulos, W. G. Johnson, A. M. Lazzarini, R. C. Duvoisin, G. Di Iorio, L. I. Golbe and R. L. Nussbaum, Mutation in the alpha-synuclein gene identified in families with Parkinson's disease, Science 276 (1997), pp. 2045-2047.
Reiber, 2003 H. Reiber, Proteins in cerebrospinal fluid and blood: barriers, CSF flow rate and source-related dynamics, Restor Neurol. Neurosci. 21 (2003), pp. 79-96.
Rimm, 2006 D. L. Rimm, What brown cannot do for you, Nat. Biotechnol. 24 (2006), pp. 914-916.
Scherzer et al., 2007 C. R. Scherzer, A. C. Eklund, L. J. Morse, Z. Liao, J. J. Locascio, D. Fefer, M. A. Schwarzschild, M. G. Schlossmacher, M. A. Hauser, J. M. Vance, L. R. Sudarsky, D. G. Standaert, J. H. Growdon, R. V. Jensen and S. R. Gullans, Molecular markers of early Parkinson's disease based on gene expression in blood, Proc. Natl. Acad. Sci. U.S.A. 104 (3) (2007), pp. 955-960.
Schlossmacher, 2007 M. Schlossmacher, Alpha-synuclein and synucleinopathies, The Dementias 2, Butterworth-Heinemann, Oxford (2007), pp. 184-213.
Schlossmacher, 2006 M. G. Schlossmacher, Monitoring of alpha-synuclein steady-state levels in vivo by ELISA, Movement disorders 21 (026) (2006).
Schlossmacher and Shimura, 2005 M. G. Schlossmacher and H. Shimura, Parkinson's disease: assays for the ubiquitin ligase activity of neural Parkin, Methods Mol. Biol. 301 (2005), pp. 351-369.
Seubert et al., 1992 P. Seubert, C. Vigo-Pelfrey, F. Esch, M. Lee, H. Dovey, D. Davis, S. Sinha, M. Schlossmacher, J. Whaley and C. Swindlehurst et al., Isolation and quantification of soluble Alzheimer's beta-peptide from biological fluids, Nature 359 (1992), pp. 325-327.
Shin et al., 2005 Y. Shin, J. Klucken, C. Patterson, B. T. Hyman and P. J. McLean, The co-chaperone carboxyl terminus of Hsp70-interacting protein (CHIP) mediates {alpha}-synuclein degradation decisions between proteasomal and lysosomal pathways, J. Biol. Chem. 280 (2005), pp. 23727-23734.
Shtilerman et al., 2002 M. D. Shtilerman, T. T. Ding and P. T. Lansbury Jr., Molecular crowding accelerates fibrillization of alpha-synuclein: could an increase in the cytoplasmic protein concentration induce Parkinson's disease?, Biochemistry 41 (2002), pp. 3855-3860.
Singleton et al., 2003 A. B. Singleton, M. Farrer, J. Johnson, A. Singleton, S. Hague, J. Kachergus, M. Hulihan, T. Peuralinna, A. Dutra, R. Nussbaum, S. Lincoln, A. Crawley, M. Hanson, D. Maraganore, C. Adler, M. R. Cookson, M. Muenter, M. Baptista, D. Miller, J. Blancato, J. Hardy and K. Gwinn-Hardy, Alpha-synuclein locus triplication causes Parkinson's disease, Science 302 (2003), p. 841.
Spillantini et al., 1997 M. G. Spillantini, M. L. Schmidt, V. M. Lee, J. Q. Trojanowski, R. Jakes and M. Goedert, Alpha-synuclein in Lewy bodies, Nature 388 (1997), pp. 839-840.
Stefanis et al., 2001 L. Stefanis, K. E. Larsen, H. J. Rideout, D. Sulzer and L. A. Greene, Expression of A53T mutant but not wild-type alpha-synuclein in PC12 cells induces alterations of the ubiquitin-dependent degradation system, loss of dopamine release, and autophagic cell death, J. Neurosci. 21 (2001), pp. 9549-9560.
Tokuda et al., 2006 T. Tokuda, S. A. Salem, D. Allsop, T. Mizuno, M. Nakagawa, M. M. Qureshi, J. J. Locascio, M. G. Schlossmacher and O. M. El-Agnaf, Decreased alpha-synuclein in cerebrospinal fluid of aged individuals and subjects with Parkinson's disease, Biochem. Biophys. Res. Commun. 349 (2006), pp. 162-166.
van Geel et al., 2008 W. J. van Geel, W. F. Abdo, R. Melis, S. Williams, B. R. Bloem and M. M. Verbeek, A more efficient enzyme-linked immunosorbent assay for measurement of alpha-synuclein in cerebrospinal fluid, J. Neurosci. Methods 168 (2008), pp. 182-185.
Wadsworth et al., 2006 J. D. Wadsworth, S. Joiner, J. M. Linehan, S. Cooper, C. Powell, G. Mallinson, J. Buckell, I. Gowland, E. A. Asante, H. Budka, S. Brandner and J. Collinge, Phenotypic heterogeneity in inherited prion disease (P102L) is associated with differential propagation of protease-resistant wild-type and mutant prion protein, Brain 129 (2006), pp. 1557-1569.

1.-50. (canceled)

51. A method to determine whether a subject has a likelihood to develop a neurodegenerative disease, or for diagnosing a neurodegenerative disease in the subject, which comprises:
quantifying the amount of total alpha-synuclein and of total protein in a cerebrospinal fluid (CSF) sample obtained from the subject and calculating a ratio of total alpha-synuclein to total protein content;
determining the age of the donor;
comparing the ratio of total alpha-synuclein to total protein content in the CSF sample obtained from the subject with the alpha-synuclein to total protein content ratio in CSF reference samples obtained from medically healthy and from neurologically healthy subjects from the same age range that at the time of sampling did not show clinical signs of neurodegenerative disease; and
determining, from the comparing step, whether the subject has a likelihood to develop neurodegenerative disease or making a diagnosis of neurodegenerative disease in the subject, whereby a difference in the ratio of total alpha-synuclein to total protein content in the CSF sample of the subject when compared with the ratio of alpha-synuclein to total protein content in the CSF reference samples obtained from the medically healthy and neurologically healthy subjects from the same age range indicates that the subject has a likelihood to develop a neurodegenerative disease or has developed a neurodegenerative disease.

52. The method according to claim 51, which further comprises quantifying the amount of total tau protein in a cerebrospinal fluid CSF sample from the subject and determining whether the subject has a likelihood to develop neurodegenerative disease or making a diagnosis of neurodegenerative disease within the group of Alzheimer disease and Alzheimer-like illnesses in a subject, whereby a rise in the content of total tau protein in the CSF sample of the subject when compared with the total tau (and/or phosphor-tau) level in the CSF samples obtained from neurologically healthy subjects from the same age range indicates that the subject has a likelihood to develop a neurodegenerative disease or has developed a neurodegenerative disease with Alzheimer disease-type pathology.

53. The method according to claim 51, further comprising quantifying the amount of total alpha-synuclein in a sample of plasma and/or whole blood obtained from the subject, determining the complete blood count and calculating a ratio of plasma alpha-synuclein to reticulocyte count and a ratio of alpha-synuclein to the aggregate hematological factor, and/or a ratio of whole blood alpha-synuclein to reticulocyte count and a ratio of alpha-synuclein to the aggregate hematological factor;
wherein a reduction in the ratio of plasma alpha-synuclein to reticulocyte content and plasma alpha-synuclein to aggregate hematological factor content in the sample from the subject indicates an increased likelihood of developing Parkinson disease, dementia with Lewy bodies or other alpha-synuclein related neurodegenerative disease; and a rise in the ratio of whole blood alpha-synuclein to reticulocyte content and whole blood alpha-synuclein to aggregate hematological factor content in the blood sample from the subject indicates an increased correlation in the likelihood of developing Parkinson disease, dementia with Lewy bodies or other alpha-synuclein related neurodegenerative disease.

54. The method according to claim 51, which further comprises:
quantitating the amount of EDTA whole blood lysate alpha-synuclein and the complete blood count in a blood sample obtained from the subject and calculating a ratio of EDTA whole blood lysate alpha-synuclein to reticulocyte count in the blood sample and/or the ratio of alpha-synuclein to the aggregate hematological factor in the blood sample;
comparing the ratio of alpha-synuclein to reticulocyte or aggregate hematological factor in the blood sample obtained from the subject with the alpha-synuclein to reticulocyte or aggregate hematological factor ratio in blood samples obtained from healthy subjects that at the time of sampling did not show clinical signs of neurodegenerative disease and that did not develop neurodegenerative disease; and
comparing the ratio of alpha-synuclein to reticulocyte or aggregate hematological factor in the blood sample obtained from the subject with the alpha-synuclein to reticulocyte, or aggregate hematological factor ratio in blood samples obtained from neurological subjects that at the time of sampling did not show clinical signs of a neurodegenerative disease linked to intracellular alpha synuclein aggregation in the brain and that did not develop neurodegenerative disease linked to intracellular alpha synuclein aggregation;
wherein a reduction in the ratio of plasma alpha-synuclein to reticulocyte content and plasma alpha-synuclein to aggregate hematological factor content in the sample from the subject indicates an increased likelihood of developing Parkinson disease, dementia with Lewy bodies or alpha-synuclein related neurodegenerative disease; and a rise in the ratio of whole blood alpha-synuclein to reticulocyte content and whole blood alpha-synuclein to aggregate hematological factor content in the blood sample from the subject indicates an increased correlation in the likelihood of developing Parkinson disease, dementia with Lewy bodies or other alpha-synuclein related neurodegenerative disease.

55. The method according to claim 51, wherein the neurodegenerative disease is a alpha-synuclein related disorder selected from sporadic Parkinson disease/Parkinsonism, familial Parkinson disease/Parkinsonism, sporadic or heritable Dementia with Lewy-Bodies, multiple system atrophy, Alzheimer's disease variants with Lewy body pathology, Down's syndrome variants with Lewy bodies, essential tremor with Lewy bodies, pure autonomic failure and neuropathy with synuclein deposition, incidental Lewy body disease associated with advanced age, lysosomal storage disorder with alpha-synuclein deposition, hereditary neurodegeneration with brain iron accumulation, familial Parkinson disease/Parkinsonism with dementia resulting from mutant genes, secondary Parkinson disease/Parkinsonism resulting from neurotoxin exposure/drug-induced Parkinsonism with alpha-synuclein deposition, Gaucher's disease with associated Parkinsonism, and conditions associated with central and/or peripheral alpha-synuclein accumulation in mammals, or the neurodegenerative disease is a disorder related to progressive neuronal damage selected from Creutzfeldt-Jakob disease and other prion diseases, or the neurodegenerative disease is an acute neurological disease selected from meningitis/encephalitis, acute cerebral ischemia /hemorrhage, and head trauma.

56. The method according to claim 51, wherein the neurodegenerative disease is Parkinson's disease (PD), dementia with Lewy bodies (DLB), multiple system atrophy (MSA), Alzheimer disease (AD), Creutzfeldt-Jakob disease (CJD), Bovine spongiform encephalopathy (BSE) or other prion disease.

57. The method according to claim 51, which is conducted for early diagnosis of the neurodegenerative disease.

58. The method according to claim 51, wherein the amount of alpha-synuclein in the CSF sample is quantitated using an enzyme-linked immunosorbent assay (ELISA) method.

59. The method according to claim 58, wherein the ELISA method is carried out using an antibody specific for alpha-synuclein.

60. The method according to claim 59, wherein the antibody specific for alpha-synuclein is biotinylated and detected using enzyme-linked avidin.

61. The method according to claim 59, wherein the antibody is biotinylated with twice the normal concentration of biotin, and the enzyme-linked avidin is ExtrAvidin alkaline phosphatase.

62. The method according to claim 51, wherein the CSF sample is not pre-concentrated.

63. The method according to claim 51, wherein a decrease in the ratio of alpha-synuclein to total protein in the CSF sample from the subject is correlated with a diagnosis of, or likelihood of developing Parkinson disease, dementia with Lewy Bodies or other alpha-synuclein related neurodegenerative disease.

64. The method according to claim 51, wherein the subject is a mammal.

65. The method according to claim 64, wherein the subject is a human and the synucleinopathy is Parkinson's disease (PD), dementia with Lewy bodies (DLB), multiple system atrophy (MSA), Alzheimer disease (AD) or Creutzfeldt-Jakob disease (CJD).

66. The method according to claim 64, wherein the subject is a bovine mammal and the synucleinopathy is Bovine Spongiform Encephalopathy (BSE) or other prion disease.

67. The method according to claim 59, wherein the anti-alpha-synuclein antibody is a monoclonal alpha-synuclein antibody.

68. The method according to claim 51, wherein the CSF is prepared in the presence of at least one protease inhibitor and at least one detergent.

69. The method according to claim 68, wherein the at least one detergent is NP40.

70. A diagnostic kit for determining whether a subject has a likelihood to develop a neurodegenerative disease and/or for the diagnosis of a subject suffering from neurodegenerative disease, comprising an antibody that specifically recognizes alpha-synuclein, and instructions for quantitating the amount of alpha-synuclein and total protein content in a cerebrospinal fluid (CSF) sample obtained from the subject using the antibody and correlating a ratio of the alpha-synuclein content to the total protein content with a likelihood of developing a neurodegenerative disease and/or a diagnosis of neurodegenerative disease.


SEQUENCE LISTING (14 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 13 aa, Homo sapiens
KAKEGVVAAAEKT

SEQ ID NO: 3 - protein, 17 aa, Homo sapiens
KTKEGVVHGVATVAEKT

SEQ ID NO: 4 - protein, 15 aa, Homo sapiens
KEGVVHGVATVAEKT

SEQ ID NO: 5 - protein, 22 aa, Homo sapiens
KEQVTNVGGAVVTGVTAVAQKT

SEQ ID NO: 6 - protein, 24 aa, Homo sapiens
KTKEQVTNVGGAVVTGVTAVAQKT

SEQ ID NO: 7 - protein, 18 aa, Homo sapiens
KTVEGAGSIAAATGFVKK

SEQ ID NO: 8 - protein, 13 aa, Homo sapiens
KAKEGVVAAAEKT

SEQ ID NO: 9 - protein, 13 aa, Homo sapiens
KTKEGVLYVGSKT

SEQ ID NO: 10 - protein, 15 aa, Homo sapiens
KEGVVHGVATVAEKT

SEQ ID NO: 11 - protein, 15 aa, Homo sapiens
KEGVVHGVATVAEKT

SEQ ID NO: 12 - protein, 24 aa, Homo sapiens
KTKEQVTNVGGAVVTGVTAVAQKT

SEQ ID NO: 13 - protein, 22 aa, Homo sapiens
KEQVTNVGGAVVTGVTAVAQKT

SEQ ID NO: 14 - protein, 18 aa, Homo sapiens
KTVEGAGSIAAATGFVKK